(19) United States
(12) Patent Application Publication
(10) Pub. No.: US 2012/0252060 A1
(43) Pub. Date: Oct. 4, 2012

(54) Self-Renewing Single Human Hematopoietic Stem Cells, an Early Lymphoid Progenitor and Methods of Enriching the Same

(76) Inventors: John Dick, Toronto (CA); Faiyaz Notta, Toronto (CA); Sergei Doulatov, Boston, MA (US); Elisa Laurenti, Toronto (CA)

(21) Appl. No.: 13/500,186

(22) PCT Filed: Oct. 8, 2010
(86) PCT No.: PCT/CA2010/001625

§ 371 Date: Jun. 19, 2012

Related U.S. Application Data
(60) Provisional application No. 61/250,174, filed on Oct. 9, 2009.

(51) Int. Cl. C12N 5/0789; G01N 21/64; C12Q 1/04

(52) U.S. Cl. 435/34; 435/372

(57) ABSTRACT

This invention relates to human hematopoietic stem cells. Specifically the invention relations to the identification of single human hematopoietic stem cells capable of long-term multilineage engraftment and self-renewal. The invention also relates to an early lymphoid progenitor with monocytic potential, including dendritic cell potential.

### FIELD OF INVENTION

This invention relates to human hematopoietic stem cells. Specifically the invention relations to the identification of single human hematopoietic stem cells capable of long-term multilineage engraftment and self-renewal. The invention also relates to an early lymphoid progenitor with monocytic potential, including dendritic cell potential.

### BACKGROUND

The origins of the hierarchical organization blood system are grounded on the discovery of the colony forming unit-spleen (CFU-S) that provided irrefutable evidence that only rare cells within the bone marrow had the capacity to undergo extensive proliferation. Since then the delineation of all major cellular classes that comprise the hematopoietic system in the mouse has been enormous, and its impact uncontested. The corresponding hierarchical roadmap in human is lacking and substantial differences in the lifespan, division kinetics of stem and precursors cells, and extinction rates of mature lineages between mouse and man clearly identify the need for similar analyses of human blood. All major progenitor classes within the human hematopoietic hierarchy were recently mapped, however the earliest steps of human blood development remain poorly understood primarily due to the inability to define rare hematopoietic stem cells (HSCs) at clonal resolution. Since extensive self-renewal capacity is endowed only to HSCs that perpetually give rise progenitor intermediates that undergo commitment to one of the blood lineages, its identification in the human blood is critical for both biological and clinical purposes.

All primitive cells in human neonatal cord blood (CB) and adult bone marrow reside in the CD34⁺CD38⁻ compartment, including Thy1^{-/lo}CD45RA⁺ multi-lymphoid progenitors (MLPs) and Thy1⁺CD45RA⁻ HSCs^{50,51}. It is well known that only a small proportion of Thy1⁺cells possess the capacity to sustain extended multi-lineage hematopoiesis, which defines stem cells, however the extent of heterogeneity is unknown due to absence of limiting dilution or single cell analysis. Although there is a need for additional markers to isolate human HSC, understanding of stem cell function is also dependent on elucidation of the stages of ontogeny coincident with cessation of self-renewal, but preceding lineage restriction of MLPs. In theory, comparison of HSC versus their immediate progeny should reveal molecular networks that sustain self-renewal and facilitate the manipulation and expansion of HSCs for cellular therapies. Majeti et al. recently reported the identification of human multipotent progenitors (MPP) as a Thy1⁻CD45RA⁻ cell within the CD34⁺CD38⁻ compartment, proposing that the loss of Thy1 expression is associated with the earliest differentiation divisions of HSCs⁵¹. However, the residual long-term engraftment capacity of Thy1⁻ cells suggest that this fraction remains heterogeneous and warrants further investigation.

Blood and other highly regenerative tissues are organized as cellular hierarchies derived from multipotent stem cells. Mouse hematopoietic stem cells (HSCs) are defined as Lin⁻Sca−1⁺ c-Kit⁺ (LSK) CD150⁺ cells lacking expression of Flt3 and CD34, whereas human HSCs are enriched in the Lin⁻CD34⁺CD38⁻ compartment^{1,2}. As HSCs differentiate, they give rise to progenitor cells which undergo lineage commitment to one of ten distinct blood lineages. The popular ‘classical’ model of hematopoiesis postulates that the earliest fate decision downstream of HSCs is the divergence of lymphoid and myeloid lineages giving rise to common lymphoid progenitors (CLPs) and common myeloid progenitors CMPs)^{3,4}. However, clonal analyses showed that most LSK Flt3⁺ lymphoid-primed multipotent progenitors (MLPPs) lack erythroid and megakaryocytic (E-MK) potential indicating that these lineages branch off prior to the lymphoid-myeloid split⁵⁻⁷. The classical model predicts that CLP is the source of all lymphoid cells, and that their progeny lack myeloid lineage potential. By contrast, several lymphoid progenitors have since been isolated that are capable of giving rise to B, T, and natural killer (NK) cells. These include LSK Flt3^{hi}VCAM1⁻MLPPs⁷, c-Kit^{hi} Ragl-expressing early lymphoid progenitors (ELPs)⁸, and c-Kit⁻ B220⁺ Ptcra-expressing CLP2 progenitors⁹. Furthermore, an extensive interrogation of multi-lineage outcomes in murine fetal liver revealed that myeloid output is retained during lymphoid specification^{10, 11}, which was confirmed by the clonal analysis of c-Kit⁺ CD25⁻ earliest thymic progenitors (ETPs)^{12, 13}. According to the classical model, during T cell commitment, CLPs first undergo myeloid restriction followed by the loss of B cell potential. However, ETPs were shown to retain myeloid, but not B cell, potential in stromal co-cultures and extensively contribute to thymic granulocyte and macrophage populations^{12, 13}. Thus, lymphoid development in the mouse appears to be a gradual process marked by several progenitor intermediates which differ in the extent of their lymphoid restriction and retention of myeloid potential^{14, 15}. There is increasing consensus for revision of the classical model to account for this evidence.

### SUMMARY OF THE INVENTION

In one aspect, there is provided a method for enriching a population of cells for human hematopoietic stem cells (HSCs) comprising:
identifying and providing the population of cells that is a source of HSCs and is to be enriched for HSCs; and
sorting cells in the population by a level of CD49f expression.

According to a further aspect, there is provided a method for enriching a population of cells for human hematopoietic stem cells (HSCs) comprising:
identifying and providing the population of cells that is a source of HSCs and is to be enriched for HSCs; and
sorting cells in the population by a level of Rhodamine-123 staining.

According to a further aspect, there is provided a population of cells enriched for HSCs obtained by the methods described herein.

According to another broad aspect, there is provided a method for enriching a population of cells for multi-lymphoid progenitor cells (MLPs) comprising:
identifying and providing the population of cells that is a source of MLPs and is to be enriched for MLPs; and
sorting cells in the population by the level of Lin, CD34, CD38 and CD45RA expression.

According to a further aspect, there is provided a method for enriching a population of cells for lymphoid myeloid progenitor cells (MLPs) comprising:
identifying and providing the population of cells from umbilical cord blood mobilized peripheral blood, or bone marrow that is to be enriched for MLPs; and
sorting cells in the population by the level of Lin, CD34, CD38 and CD10 expression.

According to a further aspect, there is provided a population of cells enriched for MLPs obtained by the methods described herein.

According to a further aspect, there is provided a method for producing a population of dendritic cells comprising:
providing a population of MLPs;
expanding the population of MLPs to produce an expanded population of MLPs;
differentiating the expanded population of MLPs to produce a differentiated population of immature dendritic cells.

According to a further aspect, there is provided a population of mature dendritic cells produced by the methods described herein.

According to a further aspect, there is provided use of Rhodamine-123 for enriching a population of cells for human HSC.

According to a further aspect, there is provided use of an anti-CD49f antibody for enriching a population of cells for human HSC.

According to a further aspect, there is provided use of a population of MLPs for producing a population of dendritic cells.

### BRIEF DESCRIPTION OF THE FIGURES

Embodiments of the invention may best be understood by referring to the following description and accompanying drawings.

FIG. 1 shows HSC sorting strategy and Functional characterization of Thy1 subpopulations within CD34⁺CD38⁻CD45RA⁻ population of Lin⁻CB. A. Freshly thawed lineage depleted cord blood cells used were stained with indicated monoclonal antibodies and applied to the cell sorter (Aria II). Dead cells were precluded from analysis using a stringent FSC/SSC gate (L1) in combination with TAminoactinomycin (7⁻AAD) dye exclusion (L2). The 10 population gates (P1−P10) analyzed quantitatively in the present study were sorted to high purity (>99%). To limit variability across experiments, population gates were consistently set using unstained, fluorescence minus one (FMO) and internal controls (refer to Methods). The percentage of each sub⁻ population, presented as percent frequency from previous gate, were maintained across all experiments. Gates are indicated. Cell surface phenotype of populations in A. Abbreviations (Abb.) of Thy1⁺ and Thy1⁻ subpopulations used in the text. FSC, forward scatter; SSC, side scatter; Live, L; Population, P. B. Quantitation of B⁻ lymphoid (CD19⁺CD33⁻) and myeloid (CD33⁺CD19⁻) differentiation capacity of Thy1⁺ (n=52) and Thy1⁻ cells (n=50) in NSG mice. Data was pooled between injected femur (IF) and BM and is presented as frequency of human CD45 positive cells. C. Mean levels of human chimerism achieved after transplant of Thy1⁺ (n=65) and Thy1⁻ cells (n=30) in NSG mice. Data represents pooled analysis from 6 independent experiments. D. Mean human engraftment levels of secondary recipients transplanted with whole bone marrow from CD90⁺ and CD90⁻ primary recipients. E. Estimate of long-term repopulating cell frequency within Thy1⁺ and Thy1⁻ cells using limiting dilution analysis. F. Experimental design to evaluate reversibility of surface Thy1 expression. Freshly sorted Thy1⁺ and Thy1⁻ cells were plated on mouse OP9 stromal cell with cytokines (SCF⁺FLT3,TPO,IL⁻7). G. Cell surface expression profile of Thy1 and CD45RA antigens after 7 days of culture on mouse OP9 stromal cells. Data is representative of 5 independent experiments. H. To assess if Thy1⁻ cells can acquire Thy1 surface expression in vivo, NSG mice transplanted with Thy1⁻ cells were assessed Thy1 and CD45RA surface antigens after 20 wk transplant period (right). Similar to F, profiles are gated on CD34⁺CD38⁻ cells. Analysis is representative from pool of 5 mice (per group). To improve resolution we performed lineage depletion to remove mouse and differentiated human cells prior to analysis by flow cytometry. Thy1⁺ mice were used as a positive control (left). I. Mean levels of chimerism in NSG mice transplanted with Thy1 positive and negative cells derived after 7 day culture period on OP9 stroma from freshly sorted Thy1⁺ and Thy1⁻ populations (day 0). Data was pooled from 2 independent experiments (d0→d7: Thy1⁺→Thy1⁺, n=9; Thy1⁺→Thy1⁻, n=4; Thy1⁻→Thy1⁺, n=9; Thy1⁻→Thy1⁻, n=9) J. Short-term engraftment potential (4 wk) in NSG mice from d7 Thy1⁺ and d7 Thy1⁻ cells derived from d0 Thy1⁻ cells. Auto, Autofluorescence; d0, day 0; d7, day 7; IF, injected femur; BM, left femur ⁺ 2 tibiae; SP, Spleen; TH, Thymus.

FIG. 2 shows that female NOD/SCID/IL-2Rg_{c}^{null} mice more efficiently support human HSC detection and proliferation then syngeneic male mice. A. Representative flow cytometric analysis of human hematopoietic cells from the injected femur of male and female recipients transplanted with the identical cell dose of sorted lineage depleted cord blood (CB). B and C. Donor human chimerism (B) and fold difference in engraftment (C) for male and female NSG recipients transplanted with non-limiting HSC doses (>1 HSC). D and E. Donor human chimerism (D) and fold difference in engraftment (E) for male and female NSG recipients transplanted with a dose equivalent of a single HSC according to LDA analyses in FIG. 1F. F. Fold difference in engraftment between male and female recipients between limiting and non-limiting HSC doses for the injected femur (IF), BM, SP and TH. G. Representative flow cytometric analysis of a simultaneous secondary transplant into a single male and female donor from a female that was transplanted with sorted Lin⁻CB. H. Reanalysis of HSC frequency for CD90⁺ (left, n=24f, 19m) and CD90⁻ (right, n=19f, 4m) fractions from FIG. 1F according to sex of the recipient. I. Summary of HSC frequency for CD90⁺ and CD90⁻ fractions according to sex of the recipient. (bars represent mean. *P <0.05, ***P<0.001).

FIG. 3 shows that human HSCs are demarcated by CD49f expression. A. Thy1⁺ and Thy1⁻ cells were sorted according to CD49f expression (CD49f: P9, P4; CD49f: P8, P3) and transplanted into NSG mice. Representative flow cytometric analysis from injected femurs is displayed. B. Mean engraftment levels in IF, BM, SP and TH. C. Fold difference in engraftment between Rho^{lo} and Rho^{hi} mice. D. 5 of 8 Rho^{lo} and 3 of 4 Rho^{hi} mice transplanted were engrafted at the doses indicated. Limiting dilution analyses indicates that 1 in 10.2 in −CD90⁺Rho^{lo} cells represents an HSC. E. Mean levels of human chimerism assessed 20⁻24 wk after transplantation of CD49f subfractions of Thy1⁺ and Thy1⁻ cells in NSG mice. Data is displayed at mean ±s.e.m. from 3 independent experiments (number of mice: P9, n=37; P4, n=8; P8, n=18; P3, n=10). Levels of engraftment in IF, BM, SP and TH are displayed as a percentage of human CD45. F. Limiting dilution analysis of all subfractions used in the study. IF, injected femur; BM, left femur⁺2 tibiae; SP, Spleen; TH, Thymus.

FIG. 4 shows engraftment of single human HSCs. A. Experimental strategy utilized to sort and transplant single P10 (Thy1⁺RholoCD49f⁺) cells. B. Human engraftment in the injected femur (IF) and BM from 3 representative NSG mice transplanted with single cells from P10 subfraction. C. Transplant efficiency from 2 independent cord blood samples. Lower efficiency from CB (2) was likely due to dramatically lower Thy1 expression levels observed at thaw. D. Mean levels of human chimerism in the IF and BM from single P10 cells. E. Analysis of Blymphoid (CD19⁺) and myeloid (CD33⁺) from D. Engraftment was also detected in the spleen, and thymus in rare cases.

FIG. 5 shows accurate detection of low level of human engraftment in NSG mice. Bone marrow from the injected femur or non-injected bones was stained with 2 separate human CD45 clones (clone J.33—Coulter, clone H130—BD) and analyzed by flow cytometry. Costaining with human specific CD19 and/or CD33 verified the human lineage being detected.

FIG. 6 shows cell cycle analysis of various human HSC and progenitor fractions. CD90⁺, CD90⁻, CD90^{lo/-}CD45RA⁺ (ELP) and CD34⁺CD38⁺ cells were sorted and processed for cell cycle (G₀, G₁ and G₂SM) analysis using Ki-67 and 7-AAD.

FIG. 7 shows Integrin expression profiling of CD90⁺ and CD90⁻ cells. Mean fluorescence intensity (MFI) of CD90⁺ and CD90⁻ cells for various markers was assessed by flow cytometry.

FIG. 8 shows the analysis of engraftuient after transplantation of human CMPs. Human CMPs (Lin⁻CD34⁺CD38⁺CD135⁺CD45RA⁻CD7⁻CD10⁻) were sorted and transplanted intrafemorally into immune-deficient recipients. Mice were sacrificed 2-4 wk post transplanted and analyzed for human cells in the injected femur (IF), BM, SP and TH. Representative flow cytometric analysis of an engrafted mice is shown above.

FIG. 9 shows the analysis of human engraftment in the injected femur (IF) and non-injected bones (BM) of single −CD90⁺Rho^{lo}CD49f^{int/hi} cells. Flow cytometric analysis of all engrafted mice is shown. Mice were sacrificed 18 wk post transplant and analyzed for human cells by using 2 non-competing human CD45 clones, CD 19 (B-cell), and CD33 (myeloid).

FIG. 10 shows the sorting scheme for human progenitors. Cord blood mononuclear cells were lineage-depleted and stained with antibodies against CD34, CD38, CD90 (Thy1), CD135 (FLT3), CD45RA, CD7, and CD10. Fraction are labeled A-G corresponding to Table 1. The proportion of cells in each gate (as % of Lin⁻ CB) is indicated next to the arrows and each of the fractions. A. Top: CD34⁺CD38^{+/hi} (CD7⁻) population was gated on FLT3, CD10 and CD45RA separating FLT3⁺CD45RA⁻ CMPs (fraction D), FLT3⁺CD45RA⁺CD10⁻ GMPs (fraction E), FLT3⁻CD45RA⁻ MEPs (fraction F), and FLT3⁺CD45RA⁺CD10⁺ pre-BINK (fraction G). Bottom: CD34⁺CD38⁻ compartment was separated based on Thy1 and CD45RA to distinguish Thy 1⁺CD45RA⁻ HSCs, Thy1⁻CD45RA⁻ MPPs (fraction A), and Thy1^{-/lo}CD45RA⁺ MLPs. Thy1^{-/lo}CD45RA⁺ fraction was further sub-gated on CD7 and CD10 (fractions B, C). The profile of Lin⁻ BM was virtually identical, except fraction C which is not found in BM (bottom right panels). B. Expression of FLT3 by human HSCs, MPPs (fraction A), and MLPs (fractions B and C).

FIG. 11 shows the clonal analysis of candidate CB and BM progenitor fractions; see Table 1 for progenitor labeling. A. Representative examples of flow cytometric determination of multi-lineage outputs in individual wells that were seeded with single CB MPPs (fraction A) and cultured for 4 wks on MS-5 stroma with SCF, TPO, IL-7, and IL-2. Only CD45⁺ events are shown. B. and C. Cloning efficiency of myeloid (left bar graph) and lymphoid (right graph) lineages of single CB B. or BM C. progenitors (labeled as fractions A-G) deposited by flow sorting onto the MS-5 stroma. The height of each bar indicates total cloning efficiency of which the proportion of myeloid (myeloid plus mixed colonies) or lymphoid (lymphoid plus mixed colonies) potential is filled in black. Morphology of cells isolated from single wells was used to validate lineage assignment (right panel, fraction B). D. T cell potential of CB (left; at 8 wks) or BM (right; at 4 wks) progenitors seeded at limiting dilution on OP9-DL1 stroma. Data are shown as limiting dilution frequency±lower and upper limits of the 95% confidence interval. E. Colony-forming efficiency of myeloid and erythroid lineages of single CB and BM progenitors deposited by flow sorting into CFU assays. Colony types: granulocytic (G), macrophage (M), mixed myeloid (GM), erythroid (E), and myelo-erythroid (GEMM). Middle panel: Giemsa stain of MLP and GMP colonies. Right panel: colony-forming (CFU-M) efficiency of CB MLPs and HSCs cultured for 4 d on OP9 stroma. Unless otherwise stated, data are shown as mean±s.e.m. of 3 independent CBs, with >12 wells for each fraction per experiment.

FIG. 12 shows the clonal analysis of human multi-lymphoid progenitors (MLPs). A. Cloning efficiency of myeloid (left bar graph) and lymphoid (right bar graph) lineages of single CB progenitors deposited by flow sorting onto MS-5 stroma and cultured for 4 wks with SCF, TPO, IL-7, IL-2, G-CSF and GM-CSF, with or without M-CSF. The height of each bar indicates total cloning efficiency of which the proportion of myeloid (myeloid plus mixed colonies) or lymphoid (lymphoid plus mixed colonies) potential is filled in black. Right panel: flow plots of representative MLP colonies. B. Cloning efficiency of T and myeloid lineages of single CB or BM MLPs or CMPs deposited by flow sorting onto MS-5-MS-5 Delta-like 4 mixed stroma and cultured for 4 wks. The height of each bar indicates total cloning efficiency of which the proportion of myeloid (myeloid plus mixed colonies) or T cell (T cell plus mixed colonies) potential is filled in black. C. Cloning efficiency of monocyte and DC lineages of single CB progenitors deposited by flow sorting onto OP9 stroma and cultured for 2 wks with GM-CSF, M-CSF, IL-4 and IL-6. Marker profiles of 4 representative MLP colonies and cell morphology of sorted Giemsa-stained CD 14⁺ and CD1a⁺ cells are shown. The height of each bar indicates total cloning efficiency of which the proportion of colonies containing both monocytes and DCs is shaded in black. All data are shown as mean ±s.e.m. of 3 independent CBs, with >12 wells for each fraction per experiment. D. The dominant transcriptional patterns observed across human HSC and progenitors recapitulate the hierarchical structure determined through functional assays. E. Validation of the expression of candidate genes by qRT-PCR.

FIG. 13 shows the differentiation of human progenitors into mature dendritic cells. Phenotypic A. and morphological B. characterization of progenitor-derived DCs. Differentiated CB MLPs, GMPs and PBMs isolated by leukopheresis were matured with IFNγ and LPS or without TLR ligands (‘No stim’). C. Proportion of mature CD80⁺ CD83⁺ CD86⁺ CD40⁺ DCs in cultures of CB MLPs, GMPs, and PBMs, matured in the presence of various cytokines and TLR ligands. D. Total expansion of CB- and BM-derived MLPs and GMPs cultured using DC conditions. E. ELISA of IL-12 (left panel) and IL-6 (right panel) secretion by DCs from MLPs, GMPs, or PBMs.

FIG. 14 shows the in vivo lineage potential of human progenitors. A. Human cell engraftment in the injected femur of NSG mice 2 wks after intra-femoral transplantation of 1,000 CB MLPs (n=4) or CMPs (n=4). Plots show graft composition gated on human CD45⁺ events. B. Representative assessment of the progenitor compartment in NSG mice 10 wks after transplantation with 100,000 Lin⁻ CB cells. Human Lin⁻ cells were isolated by column purification from the marrow of 4-8 mice, and stained with the same marker panel as in FIG. 1 without CD135 (as a result, CMP-MEP appear as a single population). C. Cloning efficiency of myeloid (left bar graph) and lymphoid (right bar graph) lineages of human progenitor fractions isolated from the bone marrow of NSG mice with human engraftment. Single cells from the indicated populations were deposited by flow sorting on MS-5 stroma and cultured for 4 wks with SCF, TPO, IL-7, and IL-2, as in FIG. 2B. Data are shown as mean±s.e.m. of 3 independent experiments, 4-8 mice each, >12 wells for each fraction per experiment.

FIG. 15 shows the lineage-specific gene expression in human progenitors. Expression of SPI1 (PU.1), CEBPA, MPO, GATA1, PAX5, and GATA3 mRNA analyzed by qPCR in progenitor fractions isolated from Lin⁻ CB by flow sorting. Data are combined from two independent experiments and plotted on a linear scale as mean±s.e.m.

FIG. 16 shows the gene expression analysis of HSC enriched subsets. A. Hierarchical clustering analysis of HSC and progenitor subsets of the human hematopoietic hierarchy using Pearson correlation coefficient with complete linkage. RNA isolated from 3 independent cord blood replicates was used for the analysis. Progenitor subsets have been defined by Doulatov et al. B. Gene expression levels of 163 HSC enriched gene-set spanning the hematopoietic hierarchy (Top). Detailed analysis of gene expression levels of transcription factors present in HSC enriched gene-set across various hematopoietic subsets (bottom). C. Go-annotation by molecular function of HSC enriched gene set. D. Summary table of gene-families of HSC gene set. E. Gene-interaction map of HSC gene set using Interologous Interaction Database (I2D). MLP, multi-lymphoid progenitor; GMP, granulocyte-macrophage progenitor; CMP, common myeloid progenitor; MEP, megakaryocyte-erythroid progenitor.

FIG. 17 shows the identification of human MPPs. A. Kinetic analysis of peripheral blood (PB) of NSG mice transplanted with CD49f subpopulations of Thy1⁺ and Thy1⁻ cells (49f⁺⁻P9, P4; 49f⁻-P8, P3). B. Representative mice were sacrificed at 2 (top) and 4 wk (bottom) after transplanted of populations indicated in A. Erythroid (GlyA⁺CD45⁻) and non⁻erythroid (CD45⁺) engraftment is shown using contour plots. C. Quantitation of total engraftment (erythroid, open; non-erythroid, closed) shown in B. Erythroid (open) and D. Absolute number of total number of human cells present in the injected femur 2 weeks after transplant. E. Schematic of major cellular classes of human hematopoietic hierarchy. All data is presented as mean±s.e.m. from n=4 recipients per group. IF, injected femur; BM, non injectedbone marrow.

### DETAILED DESCRIPTION

To date, the ability to functionally characterize and assay single human hematopoietic stem cells (HSCs) has not been achieved as most existing analyses have utilized highly heterogeneous populations in which HSCs represent a negligible fraction. Using transplantation into NOD-scid IL2Rgc^{-/-} mice, we identify CD49f as a novel marker of human HSCs. Up to 30% of CD34⁺CD38⁻CD45RA⁻Thy1⁺CD49f^{hi} cells sorted on low rhodamine-123 retention had long-term engraftnient capacity at single cell resolution. Remarkably, loss of CD49f expression simultaneously demarcated human multi-potent progenitors from HSCs and indicate that Thy1⁻ cells within CD34⁺CD38⁻CD45RA⁻ compartment remain heterogeneous. Together with Doulatov et al., these studies communicate the first comprehensive roadmap of the major cellular classes that comprise the human blood system.

Further, the classical model of hematopoiesis posits the segregation of lymphoid and myeloid lineages as the earliest fate decision. The validity of this model has recently been questioned in the mouse, however little is known concerning lineage potential of human progenitors. There is provided herein, analysis of the human hematopoietic hierarchy by clonally mapping the developmental potential of 7 progenitor classes from neonatal cord blood and adult bone marrow. Human multi-lymphoid progenitors, identified as a distinct population of Thy1^{-/lo}CD45RA⁺ cells within the CD34⁺CD38⁻ stem cell compartment, gave rise to all lymphoid cell types, as well as monocytes, macrophages, and dendritic cells, indicating that these myeloid lineages arise in early lymphoid lineage specification. Thus, as in the mouse, human hematopoiesis does not follow a rigid model of myeloid-lymphoid segregation.

In contrast with the mouse, definitive evidence for a comprehensive model that best describes human hematopoiesis is lacking. Progress has been limited by two important factors—paucity of cell surface markers used to distinguish pure populations, and the absence of assays that detect multi-lineage outputs from single cells with high cloning efficiency. Human CMPs were isolated as CD34⁺CD38⁺ IL-3Rα⁺CD45RA⁻ cells from adult bone marrow (BM), but their lineage potential at the clonal level was evaluated only using colony assays¹⁶. The earliest steps of human lymphoid development remain poorly understood. Human CLPs have been first isolated from BM as Lin⁻ CD34⁺CD10⁺ cells, only ˜3% of which gave rise to B and NK cells, but not myeloid or erythroid progeny, in clonal plating on stromal co-cultures¹⁷. Further separation of this population into CD24⁺ and CD24⁻ cells revealed that all CLP potential resided in the CD34⁺CD10⁺ CD24⁻ fraction in neonatal cord blood (CB) and BM, but the cloning efficiency remained <5%¹⁸. Other reports suggested that, at least in CB, CLPs were CD7⁺ rather than CD10⁺, and resided in the CD34⁺CD38⁻ fraction (cloning efficiency <5%)^{19, 20}. These studies failed to detect myeloid potential in the candidate CLP fractions leading to the assumption that the classical model best describes human hematopoiesis. The existence of at least some cells with multi-lymphoid progenitor (MLP) potential, defined as any progenitor minimally capable of giving rise to B, T, and NK cells, within the sorted populations is thus established. However, given low cloning efficiencies and the absence of single cell analysis, the lineage potential of rare human MLPs in these fractions cannot be conclusively assessed.

To this end, Applicant isolated 7 distinct progenitor classes from CB and BM samples based on a single panel of 7 markers and interrogated their developmental potential using clonal analysis under conditions that provided robust support of multiple lineage fates. By assembling such a comprehensive ‘roadmap’, we identified human MLPs as a distinct Thy1^{-/lo}CD45RA⁺ population within the CD34⁺CD38⁻ HSC compartment. We show that MLPs generate all lymphoid cell types, as well as monocytes, macrophages and dendritic cells, prompting a revision to the model by which human blood lineages are specified from HSCs.

In one aspect, there is provided a method for enriching a population of cells for human hematopoietic stem cells (HSCs) comprising:
identifying and providing the population of cells that is a source of HSCs and is to be enriched for HSCs; and
sorting cells in the population by a level of CD49f expression.

Preferably, the method further comprises dividing the cells into high and low Rhodamine-123 staining groups and preferably selecting for a sub-population of cells comprising the low Rhodamine-123 staining group.

In some embodiments, the method further comprises sorting the cells by the level of CD49f expression and preferably, dividing the cells into high, intermediate and low CD49f expression groups and further preferably selecting for cells comprising at least one of the intermediate and high level CD49f expression groups, preferably the high level CD49f expression group.

According to a further aspect, there is provided a method for enriching a population of cells for human hematopoietic stem cells (HSCs) comprising:
identifying and providing the population of cells that is a source of HSCs and is to be enriched for HSCs; and
sorting cells in the population by a level of Rhodamine-123 staining.

In some embodiments, the method further comprises dividing the cells into high, intermediate and low CD49f expression groups and preferably selecting for a sub-population of cells comprising at least one of the intermediate and high level CD49f expression groups, preferably the high level CD49f expression group. Preferably, the method further comprises dividing the cells into high and low Rhodamine-123 staining groups and preferably selecting for a sub-population of cells comprising the low Rhodamine-123 staining group.

In some embodiments, the methods for enriching a population of cells for human hematopoietic stem cells (HSCs) further comprises sorting cells using at least one marker selected from the group consisting of Lin, CD34, CD38, CD90, CD45RA, and preferably selecting at least one fraction selected from the group consisting of Lin⁻, CD34⁺, CD38⁻, CD90⁺, and CD45RA⁻.

In some embodiments, the source of the population of cells is at least one of bone marrow, umbilical cord blood, mobilized peripheral blood, spleen or fetal liver.

According to a further aspect, there is provided a population of cells enriched for HSCs obtained by the methods described herein.

According to another broad aspect, there is provided a method for enriching a population of cells for lymphoid myeloid progenitor cells (MLPs) comprising:
identifying and providing the population of cells that is a source of MLPs and is to be enriched for MLPs; and
sorting cells in the population by the level of Lin, CD34, CD38 and CD45RA expression.

Preferably, the method further comprises selecting for a sub-population of cells that are Lin-, CD34+, CD38⁻ and CD45RA⁺.

In some embodiments, the method further comprises sorting cells in the population by the level of expression of at least one of CD7 and CD10 and preferably, selecting for cells in at least one of CD7⁻ and CD10⁺ fractions.

In some embodiments, the source of the population of cells is at least one of bone marrow, umbilical cord blood, mobilized peripheral blood, spleen or fetal liver.

According to a further aspect, there is provided a method for enriching a population of cells for lymphoid myeloid progenitor cells (MLPs) comprising:
identifying and providing the population of cells from umbilical cord blood mobilized peripheral blood, or bone marrow that is to be enriched for MLPs; and
sorting cells in the population by the level of Lin, CD34, CD38 and CD10 expression.

Preferably, the method further comprises selecting for a sub-population of cells that are Lin−, CD34+, CD38⁻ and CD10⁺.

Further preferably, the method further comprises sorting the cells by the level of expression of at least one of CD7 and CD45RA, and preferably selecting for cells in at least one of CD7⁻ and CD45RA⁺ fractions.

In some embodiments, the method further comprises sorting the cells by the level of expression of CD90 and preferably, selecting for cells in a CD90⁺ fraction.

According to a further aspect, there is provided a population of cells enriched for MLPs obtained by the methods described herein.

According to a further aspect, there is provided a method for producing a population of dendritic cells comprising:
providing a population of MLPs;
expanding the population of MLPs to produce an expanded population of MLPs;
differentiating the expanded population of MLPs to produce a differentiated population of immature dendritic cells.

Certain methods of expanding, differentiating and maturing cells would be known to a person skilled in the art.

Preferably, the method further comprises maturing the differentiated population of immature dendritic cells to a population of mature dendritic cells.

In some embodiments, the population of MLPs is the population of cells population of cells enriched for MLPs obtained by the methods described herein.

In some embodiments, the population of MLPs is expanded on stroma, preferably selected from the group consisting of MS-5, OP9, S17, HS-5, AFT024, SI/SI4, M2-10B4 and preferably using at least one of SCF, TPO, FLT3 and IL-7.

In some embodiments, the expanded population of MLPs is differentiated using at least one of GM-CSF and IL-4.

In some embodiments, the differentiated population of immature dendritic cells is matured using at least one of IFNγ, LPS, TNFα, IL-1β, IL-6, PGE2, poly I:C, CpG, Imiquimod, LTA, IFNγ and LTA.

According to a further aspect, there is provided a population of mature dendritic cells produced by the methods described herein.

According to a further aspect, there is provided use of Rhodamine-123 for enriching a population of cells for human HSC.

According to a further aspect, there is provided use of an anti-CD49f antibody for enriching a population of cells for human HSC.

According to a further aspect, there is provided use of a population of MLPs for producing a population of dendritic cells.

As used herein, “DCs” refer dendritic cells. DCs are immune cells that form part of the mammalian immune system. Their main function is to process antigen material and present it on the surface to other cells of the immune system, thus functioning as antigen-presenting cells.

As used herein “engrafting” a stem cell, preferably an expanded hematopoietic stem cell, means placing the stem cell into an animal, e.g., by injection, wherein the stem cell persists in vivo. This can be readily measured by the ability of the hematopoietic stem cell, for example, to contribute to the ongoing blood cell formation.

As used herein, “expression” or “level of expression” refers to a measurable level of expression of the products of markers, such as, without limitation, the level of messenger RNA transcript expressed or of a specific exon or other portion of a transcript, the level of proteins or portions thereof expressed of the markers, the number or presence of DNA polymorphisms of the biomarkers, the enzymatic or other activities of the biomarkers, and the level of specific metabolites.

As used herein “hematopoietic stem cell” refers to a cell of bone marrow, liver, spleen, mobilized peripheral blood or cord blood in origin, capable of developing into any mature myeloid and/or lymphoid cell.

As used herein “stroma” refers to a supporting tissue or matrix. For example, stroma may be used for expanding a population of cells. A person of skill in the art would understand the types of stroma suitable for expanding particular cell types. Examples of stroma include MS-5, OP9, S17, HS-5, AFT024, SI/SI4, M2-10B4.

As used herein “Lineage” or “Lin” markers refer to markers that are used for detection of lineage commitment. Cells and fractions thereof that are negative for these lineage markers are therefore referred to as “Lin⁻”. As such, typically, during their purification by FACS, antibodies are used as a mixture to deplete the “Lin⁺” cells. Lineage markers include up to 14 different mature blood-lineage marker, e.g., CD13 & CD33 for myeloid, CD71 for erythroid, CD19 for B cells, CD61 for megakaryocytic, glycophorin A (glyA), CD3, CD2, CD56, CD24, CD19, CD66b, CD14 and CD16? etc. for humans; and, B220 (murine CD45) for B cells, Mac-1 (CD11b/CD18) for monocytes, Gr-1 for Granulocytes, Ter119 for erythroid cells, I17Ra, CD3, CD4, CD5, CD8 for T cells, etc. for mice.

The term “marker” as used herein refers to a gene that is differentially expressed in different cells. Examples of markers include, but are not limited to, CD13, CD33, CD71, CD19, CD61, glycophorin A (glyA), CD3, CD2, CD56, CD24, CD19, CD66b, CD14, CD16, CD49f, CD34, CD38, CD90 and CD45RA.

As used herein “sorting” of cells refers to an operation that segregates cells into groups according to a specified criterion (including but not limited to, differential staining and marker expression) as would be known to a person skilled in the art such as, for example, sorting using FACS. Any number of methods to differentiate the specified criterion may be used, including, but not limited to marker antibodies and staining dyes.

The following examples are illustrative of various aspects of the invention, and do not limit the broad aspects of the invention as disclosed herein.

### EXAMPLES

### Example 1

### Identification of Single HSCs Capable of Long-Term Multilineage Engraftment and Self-Renewal

### Methods

Lineage depleted cord blood cells were stained with the indicated antibodies prior to cell sorting. Sorted cells were transplanted into the right femur (injected femur—IF) of sublethally irradiated (200-250 cGy) NSG mice. After a minimum of 16 wks post transplant, mice were sacrificed and the injected femur (right femur), bone marrow (left femur, left and right tibiae), spleen and thymus were analyzed for human cell engraftment by flow cytometry. Statistical analysis was performed with Mann-Whitney test.

Human Cord blood. Samples of human cord blood were obtained from Trillium Hospital (Mississauga, Ontario, Canada) and processed in accordance to guidelines approved by University Health Network. Various cord blood samples were pooled and an equal volume of phosphate buffered saline was added prior to layering on Ficoll/Paque gradient (Pharmacia) in 50 mL conical tubes. Tubes were subjected to 25 min centrifugation at 400×g followed by careful removal of mononuclear layer and washed with Iscove's modified Dulbecco's medium (IMDM, GIBCO/BRL). Lineage negative cells were enriched by magnetic negative cell depletion by using human hematopoietic progenitor enrichment cocktail (Stem cell technologies, Vancouver, BC, Canada) according to manufacturer protocol. Lin− cells were stored at −150° C.

Cell preparation for cell sorting. Lin⁻ cells were thawed via the dropwise addition of IMDM+DNase (200 ug/mL final concentration) and resuspended at 10⁶ cells/mL in PBS/2.5% FBS (Sigma, St. Louis, Mo., USA). Cells were subsequently stained with CD45RA Fite or pe, CD9OPe or biotin, CD49f Pe-Cy5, CD34Apc or CD34Apc-Cy7 and CD38 Pe-Cy7 (Becton Dickinson) and incubated for 30 min at 4° C. Cells were subsequently washed with PBS/2.5% FBS and secondary staining with streptavidin-bound quantum dot 605 (Molecular Probes) was performed (30min, 4° C.) when CD90biotin conjugated antibody was used. Cells were washed again with PBS/2.5% FBS and resuspended at 10⁶-10⁷/mL in PBS/0.5% FBS prior to sort. Cells were sorted on FACS Aria (488 nm Blue [100 mW], 633 nm Red [30mW], Becton Dickinson) and collected in 1.5 mL microfuge tubes. Cells were spun down, counted via trypan blue exclusion, and resuspended in appropriate volume of PBS/0.1% FBS or IMDM for transplant. A fraction of the final volume was recounted to ensure the cell dose being transplanted was accurate. In experiments were Rhodamine 123 (Eastman Kodak, Rochester, N.Y., USA) was used, the protocol was adjusted as previously described. Briefly, freshly thawed lin⁻ cells were incubated at 37° C. with 0.1 ug/mL Rho, washed and destained at 37° C. for an additional 30 mins. Cells were subsequently subjected to staining with appropriate antibodies as mentioned above.

Single Cell transplant. Single Lin⁻CD34⁺CD38⁻CD90⁺CD45RA⁻Rho^{lo}CD49f⁺ cells were sorted into Nunc MiniTrays (163118) in 10 uL of IMDM/1% FBS or 96 well plates using the FACS Aria. Cells were allowed to settle for 1 h at 4° C. or centrifuged at 600×g for 5 min. Single cells were visualized using a microscope and transferred into a 28.5 g insulin syringe. Wells were revisualized to ensure the cell was absent after transferring into the needle. Post-sort cell viability was assessed independently using a second Minitray in which single cells were sorted. Trypan blue was added to the well and 60/60 wells analyzed had single viable cells.

Xenotransplant Assay. NOD/LtSz-scidIL2Rg^{null} (NSG) (Jackson Laboratory) were bred and housed at the Toronto Medical Discovery Tower/University Health Network animal care facility. Animal experiments were performed in accordance to institutional guidelines approved by UHN Animal care committee. The intrafemoral transplant has been previously described. Briefly, 10-12 wk old mice were irradiated (200-250 cGy) 24 h before transplant. Prior to transplantation, mice were temporarily sedated with isoflurane. A 27 g needle was used to drill the right femur (injected femur—IF), and subsequently, cells were transplanted in 25 uL volume using a 28.5 g insulin needle. For serial transplantation, IF and BM were combined and transplanted into the right femur of secondary recipients.

Assessment of human cell engraftment. All NSG mice were sacrifice>16 wk post-transplant. The right and left femur and tibiae, spleen and thymus were removed cells were extracted using standard flushing or cell dissociation techniques. Cell were then stained in PBS/2% FBS and analyzed by multiparameter flow cytometry (LSRII, Becton Dickinson) using automated compensation of anti-mouse Ig,k and negative control compensation particles (Ca. 552843, Becton Dickinson). The marrow (IF and BM) were analyzed with 2 non-competing CD45 clones (H130 PC7—Becton Dickinson, and J.33 PE or PC5—Beckman coulter). Other lineage markers used were CD3, CD4 (Beckman coulter), CD5, CD7, CD8, CD11b, CD19, CD33 (Beckman coulter), CD56, GlyA (Beckman coulter), IgM (all Becton Dickinson unless otherwise indicated).

Statistics. Data is represented as mean±s.e.m. The significance of the differences between groups was determined by using Mann-Whitney test. Limiting dilution analysis was performed using online software provided by WEHI bioinformatics (http://bioinf.wehi.edu.au/software/elda/index.html Hu, Y. and Smyth, G. (2009). ELDA: Limiting Dilution Analysis for comparing depleted and enriched populations, Walter and Eliza Hall Institute of Medical Research, Australia.)

### Results

Fractionation of HSCs based on Thy1 expression

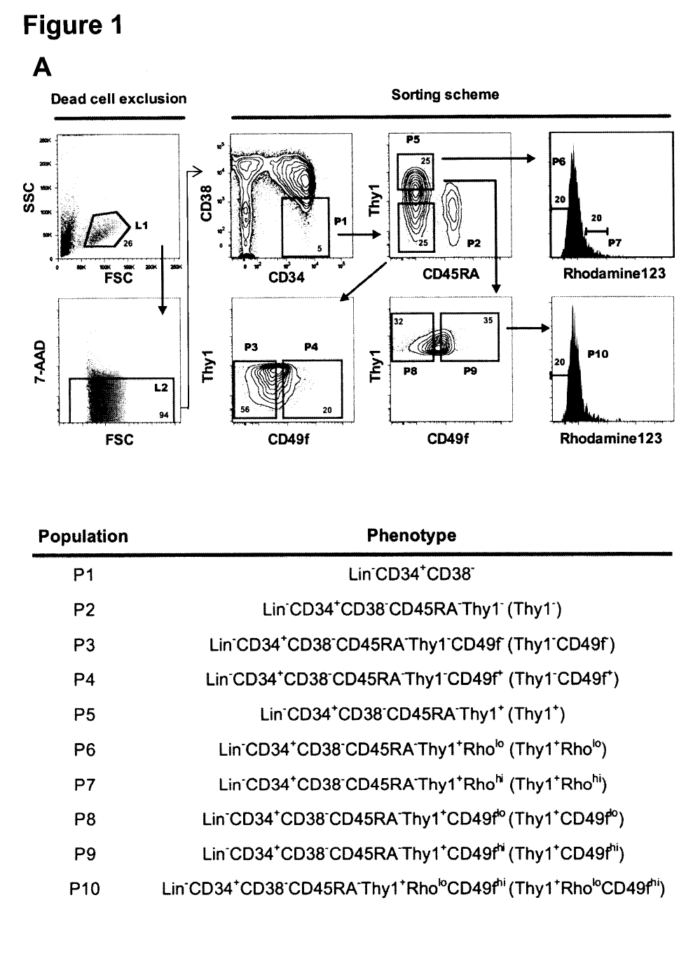

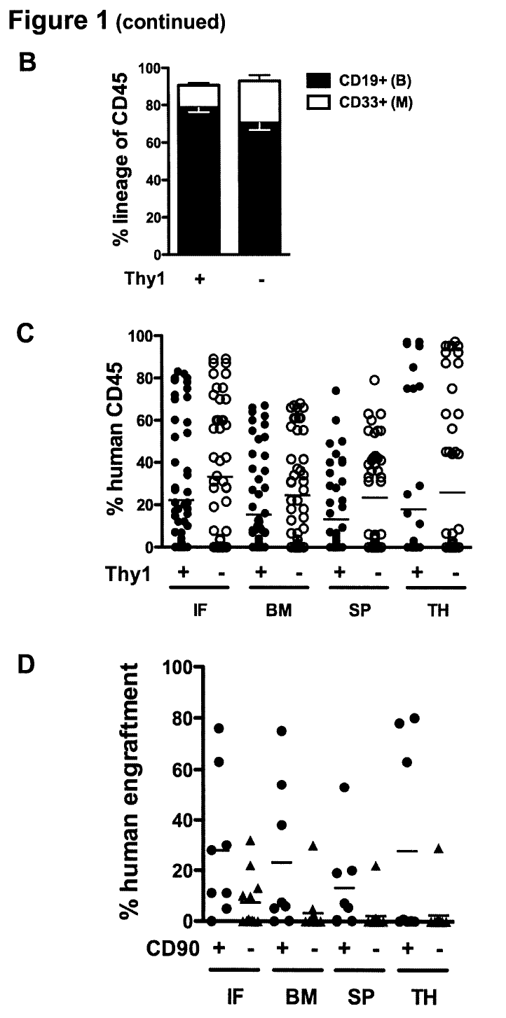

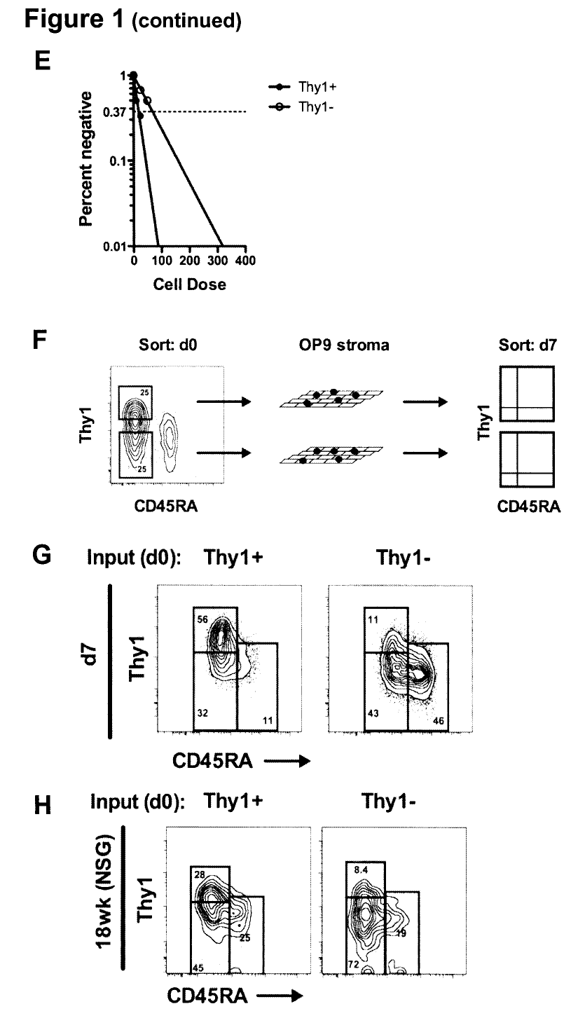

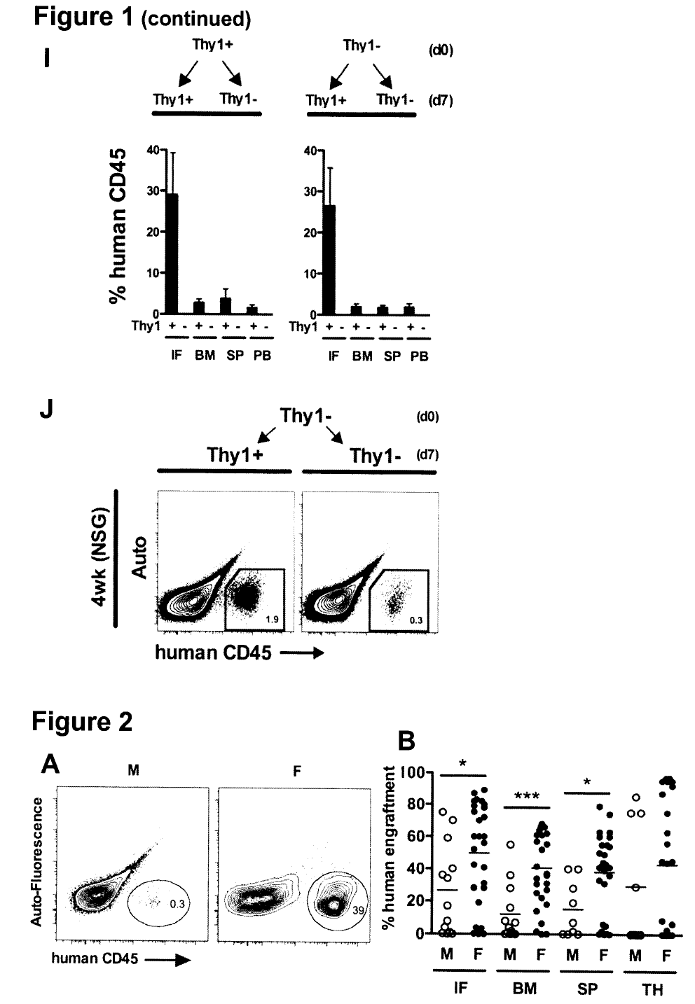

Limiting dilution (LD) analyses indicate that only 1% of CD34⁺CD38⁻ cells possess the capacity to repopulate immune-deficient mice (P1, FIG. 1A)⁵². Although a higher frequency of HSC has been proposed to exist within Thy1⁺CD45RA⁻ compartment of CD34⁺CD38⁻ cells, the extent of stem cell heterogeneity within this subfraction remains unknown in the absence of LD analysis. To directly assess the purity of the proposed human HSC (CD34⁺CD38⁻CD45RA⁻Thy1⁺, herein Thy1⁺) and MPP (CD34⁺CD38⁻ CD45RA⁻Thy1⁻, herein Thy1⁻) fractions we employed the optimized HSC assay conditions and analyzed hematopoietic reconstitution of NSG mice after 18-24 wk (FIG. 1, P2, P5). This duration encompasses both periods of primary and secondary transplant historically used to assess self-renewal capacity of human CB HSCs in xenograft models. At non-limiting cell doses, recipients of Thy1⁺ and Thy1⁺ cells had similar levels of human chimerism and lineage distribution (FIG. 1B-C). Cell cycle analysis confirmed that CD90+and CD90− cells have similar level of quiescence (FIG. 6). HSCs are distinct from MPPs in their capacity for long-term reconstitution and self-renewal upon serial transplantation⁵³. Secondary (2°) transplants were performed to determine if CD90⁺ or CD90⁻ cells represented bona fide HSCs or candidate human MPPs⁵¹. Interestingly, 7/8 and 8/12 mice transplanted with marrow from primary CD90⁺ and CD90⁻ recipients were engrafted, respectively. However, mean engraftment levels of CD90⁺ cells were much higher compared to CD90⁻, approaching statistical significance (IF: CD90⁺-28.0%, CD90⁻-7.3%, p=0.057) (FIG. 1D). We then performed LD analysis to measure the frequency of HSCs within Thy1⁺ and Thy1⁻ fractions. HSC activity was 4.2 fold higher in Thy1⁺ fraction, however about 1 in 100 Thy1⁻ also displayed a robust long-term repopulating capacity (4.9% vs. 1.1%, p=0.0003, FIG. 1E). Double sorting or high stringency sort modes and cell dose compensation implemented in our experimental design confirmed the HSC activity from Thy1⁻ cells was not due residual contamination from Thy1⁺ cells (data not shown). Therefore, this analysis reveals the frequency of human HSC within Thy1 subcompartments of CD34⁺CD38⁻CD45RA⁻ cells and suggests that HSCs remain in the absence of Thy1 expression. Thus, it was critical to test the hierarchical relationship between these populations.

### Thy1⁻ Cells Give Rise to Thy1⁺ HSCs

Previous studies have reported that Thy1⁺ HSCs give rise to Thy1⁻ MPPs that lack the capacity for sustained engraftment. To further investigate the hierarchical relationship between Thy1⁺ and Thy1⁻ cells, we cultured cells on OP9 stroma known to express ligands that support HSC (FIG. 1F). Greater then 70% of Thy1⁺ and Thy1⁻ cells remained CD34⁺CD38⁻ after 7 days of OP9 culture. Unexpectedly, Thy1⁻ cells in this condition consistently generated Thy1 positive cells (FIG. 1G). In addition, Thy1⁻ cells directly transplanted into NSG mice also gave rise to Thy1 positive cells indicating this phenomenon occurs in vivo within bone marrow (FIG. 1H). Thy1 positive cells arising from either day 0 Thy1⁻ cells, or Thy1⁺ cells that retained their expression had robust repopulating activity 20 wks after transplant into NSG mice. Engraftment and differentiation potentials were identical for Thy1 positive cells from either Thy1⁺ or arising from Thy1⁻ subfractions (FIG. 1I, p=0.93). As previously mentioned, this was consistent across several experiments even when we employed highly conservative gating or double sorting strategies (data not shown). By contrast, day 0 Thy1⁻ cells that remained Thy1⁻ did not have give rise to long-term engraftment, but did engraft short-term (4 wk, FIG. 1J). These results demonstrate that the Thy1⁻ compartment is heterogeneous and is comprised of a minor fraction that gives rise to Thy1⁺ HSCs, and a major fraction of candidate MPP-like progenitors. However, definitive evidence of this would require prospective identification using an independent marker that can segregate HSCs from MPPs.

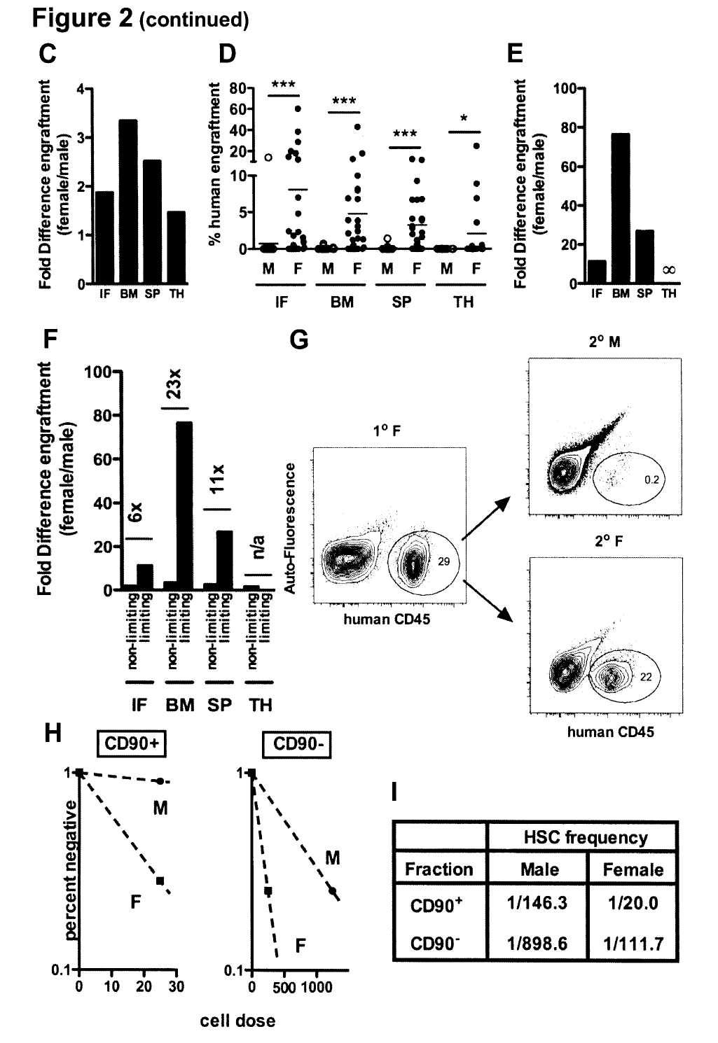

During the course of our analyses of CD90⁺ and CD90⁻ cells in NSG mice, Applicants recognized that human engraftment could be stratified according to the gender of the recipient. When multiple HSCs (non-limiting dose) were transplanted in female and male NSG mice, female mice displayed a modest but significantly higher level of human chimerism (female vs. male: IF—49.7±5.8 vs. 26.5±7.7, p=0.03; BM—40.6±4.4 vs. 12.1±4.8, p=0.0009; SP—38.1±4.5 vs. 15.1±5.7, p=0.01; TH—42.9±8.0 vs. 29.3±12.7, p=0.18; n=28 females[F], 13 males[M]) (FIG. 2A-C). However, transplantation of doses equivalent to a single HSC (limiting dose) unveiled striking differences between male and female recipients (female vs. male: IF—8.1±2.7 vs. 0.7±0.7, p=0.0001; BM—4.8±1.7 vs. 0.1±0.04, p<0.0001; SP—3.2±0.8 vs. 0.1±0.1, p<0.0001; TH—2.1±1.2 vs. 0, p=0.04; n=29F, 20M) (FIG. 2D). Female mice had 11 - and 76-fold higher mean IF and BM engraftment, respectively, compared to age-matched, syngeneic males (FIG. 2E and 2F). Next, Applicants performed parallel secondary transplants into male and female recipients from single primary females. In every case, higher levels of human engraftment were observed in female secondary recipients (FIG. 2G) providing direct evidence that human HSCs are more efficiently detected in female NSG mice. Retrospective assessment of the initial HSC frequency in female recipients revealed that in 1 in 20 CD90⁺ (n=24f) and 1 in 112 CD90⁻ (n=19f) cells is an HSC (FIGS. 2H and 2I). Further experiments are required to dissect the molecular determinants of sex-dependent difference in engraftment of human HSCs. Overall, refinement of the frequency analyses and optimization of the NSG model to detect limiting doses of HSCs sets the foundation for further purification.

### CD49f Expression Demarcates Human HSCs

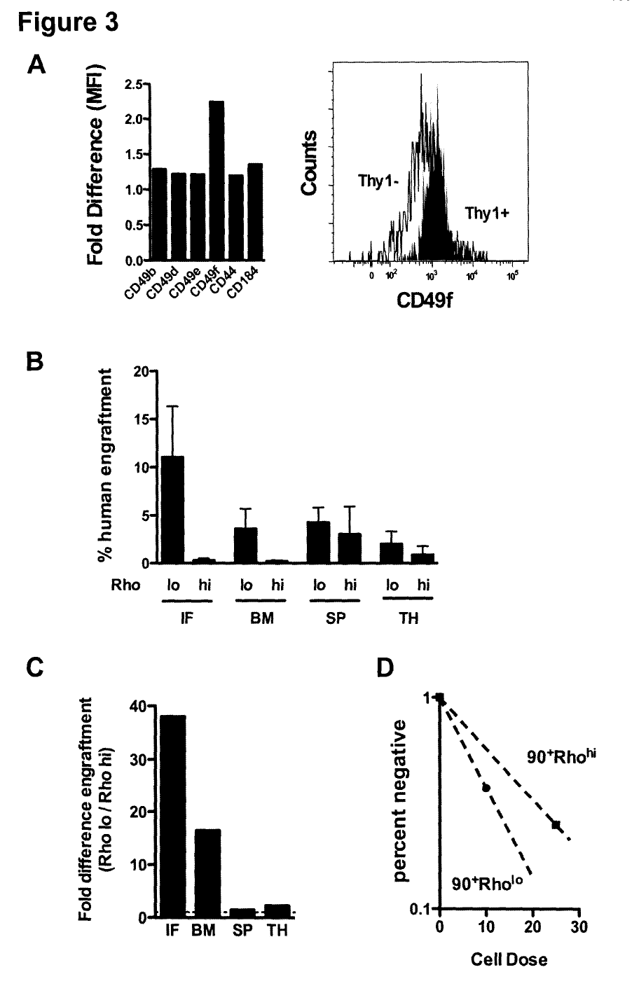

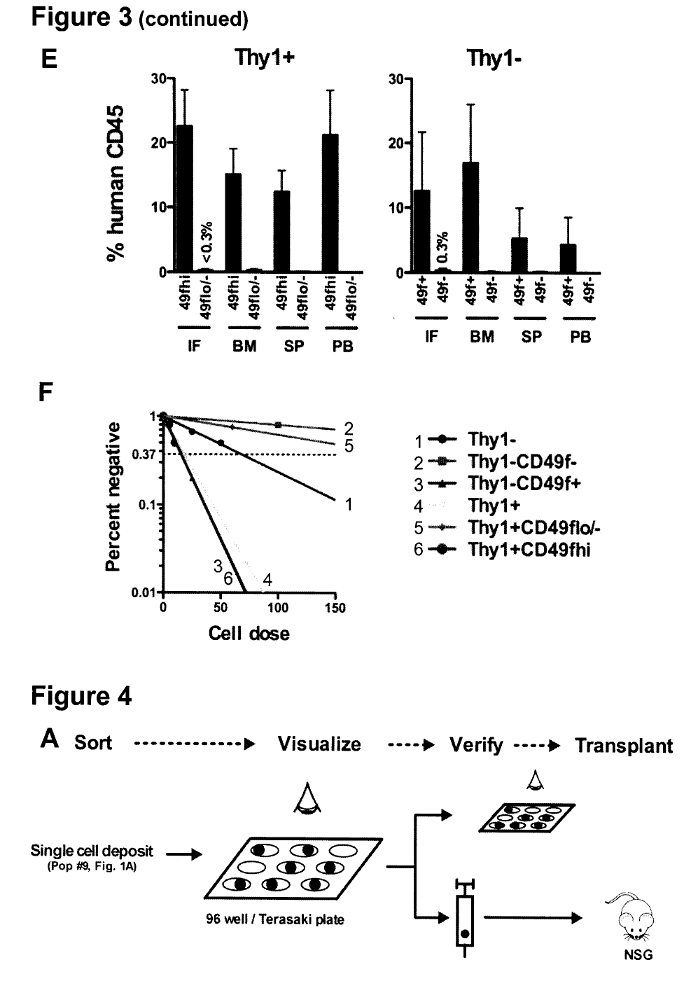

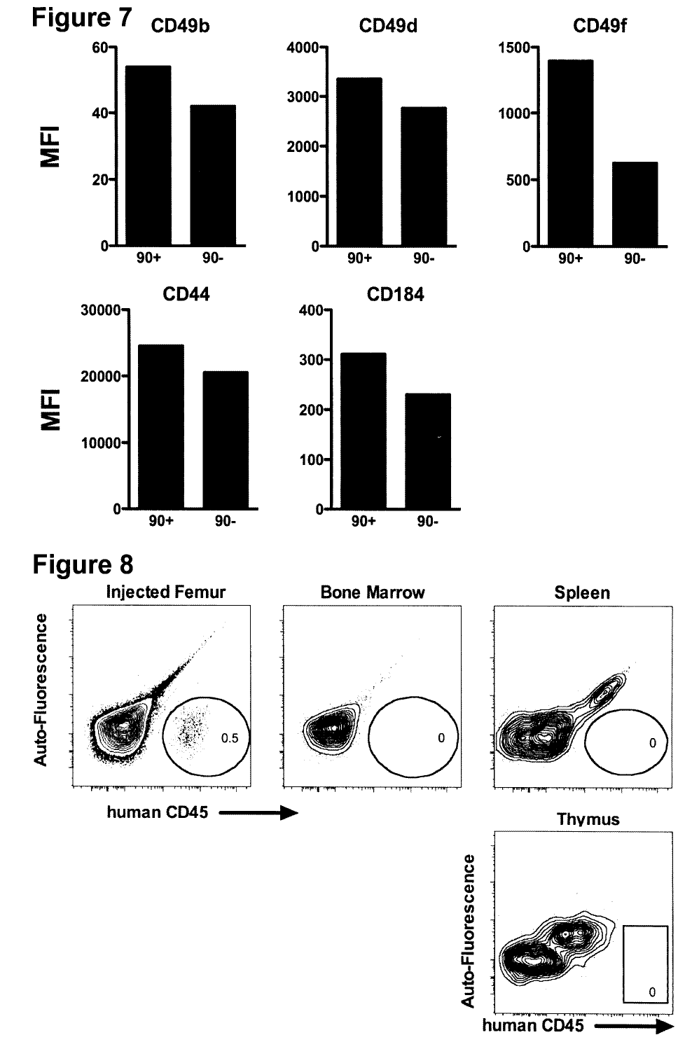

To provide direct evidence supporting the hierarchical organization of Thy1⁻ and Thy1⁺ HSC and Thy1⁻ MPP, Applicants sought to identify an independent marker that segregated HSCs from MPPs enabling prospective identification. Integrins mediate interactions between HSCs and the niche and have been used to isolate other somatic stem cells such as those from mammary epithelium. Applicants evaluated the expression of various integrins (a2, a4, a5, a6) and other molecules involved in migration, e.g. CD44 and CXCR4 (FIG. 7). We hypothesized that integrins would differentially mark human HSCs. Using flow cytometry, we compared the expression of several integrins between stem cell enriched (Thy1⁺) and depleted (Thy1⁻) fractions. Thy1⁺ cells consistently displayed a two-fold higher expression of ITGA6 (integrin α6, herein CD49f) (FIG. 3A). Fifty—70% of Thy1⁺ versus 20% of Thy 1⁻ cells were CD49f positive across multiple experiments with alternate flurochromes. Interestingly, ITGA6 is the only gene whose transcription is shared between hematopoietic, neural, and embryonic stem cells.

Cellular processes such as quiescence and energy state are closely associated with stem cell function⁵⁴⁻⁵⁶. Since mitochondria are regulators of these processes, Applicants sought to determine if the differential efflux of the mitochondrial dye, Rhodamine-123 (Rho, FIGS. 3B-3D), could be used to functionally enrich for human HSCs in combination with CD90⁺. Applicants sorted CD90⁺ fraction into Rho^{lo} and Rho^{hi} cells and transplanted them into female NSG mice. After 18 wks, CD90^{hi}Rho^{hi} mice had 40-fold higher IF engraftment compared to CD9O^{hi}ltho^{l}′ⁱ mice (FIG. 3E). Five of 8 mice transplanted with 10 CD90⁺Rho^{lo} cells versus 3 of 4 CD90⁺Rho^{hi} mice at the 25-cell dose were engrafted (FIG. 3C). Therefore, using Poisson statistics, Applicants approximate that 1 in 10.2 cells within the CD90⁺Rho^{lo} fraction is an HSC. This illustrates that the addition of Rho can enrich for HSC activity in the CD90⁺ fraction by ˜2 fold.

To test CD49f as an additional marker of HSCs, we partitioned Thy1⁺ cells into CD49f^{hi} (Thy1⁺CD49f^{hi}) and CD49f^{lo/-} (Thy1⁺CD49f^{lo/-}) subfractions and evaluated their capacity to generate long-term multilineage chimerism in NSG recipients. Mean level of chimerism in the injected femur was 86 fold higher in recipients of Thy1⁺CD49f^{hi} cells (22.6% vs. 0.3%, p<0.0001; FIG. 3E). LD analysis revealed that Thy1⁺CD49f^{hi} fraction had a 20 fold enrichment for HSC compared with Thy1⁺CD49f^{lo/- cells (}9.5% vs. 0.5%, p=5.8×10⁻⁸; FIG. 3F). Since we demonstrated that the Thy1⁻ fraction was heterogeneous, Applicants next tested whether CD49f expression also marked HSCs within this cellular subcompartment. Indeed, the recipients of Thy1⁻CD49f⁺ cells displayed long-term multi-lineage engraftment (FIG. 3D). LD analysis confirmed that it was enriched for HSC activity compared with Thy1⁻ CD49f cells (7% vs. 0.2%, p=3.6×10⁻⁵; FIG. 3F). No difference in lineage potential were observed between Thy1⁺CD49f^{hi} and Thy1⁻CD49f⁺ cells, although recipients of Thy1⁺CD49f^{hi} cells trended towards higher levels of chimerism (FIG. 3E) suggesting a higher frequency of HSCs. These data suggest that human HSCs are marked by high levels of CD49f expression, whereas Thy1 expression is not obligate.

### Engraftment of Single Human Hematopoietic Cells

Long-term and multilineage repopulation following transplantation of single cells remains the most definitive assay with which to define a stem cell as single cells must self renew to enable long-term repopulation; downstream progenitors are not able to sustain a graft long-term. Prior to this study, low frequency of repopulating cells in existing human HSC-enriched fractions made this direct test unfeasible. Applicants first tested if the low retention of mitochondrial dye, Rhodamine-123 (Rho), enriched for

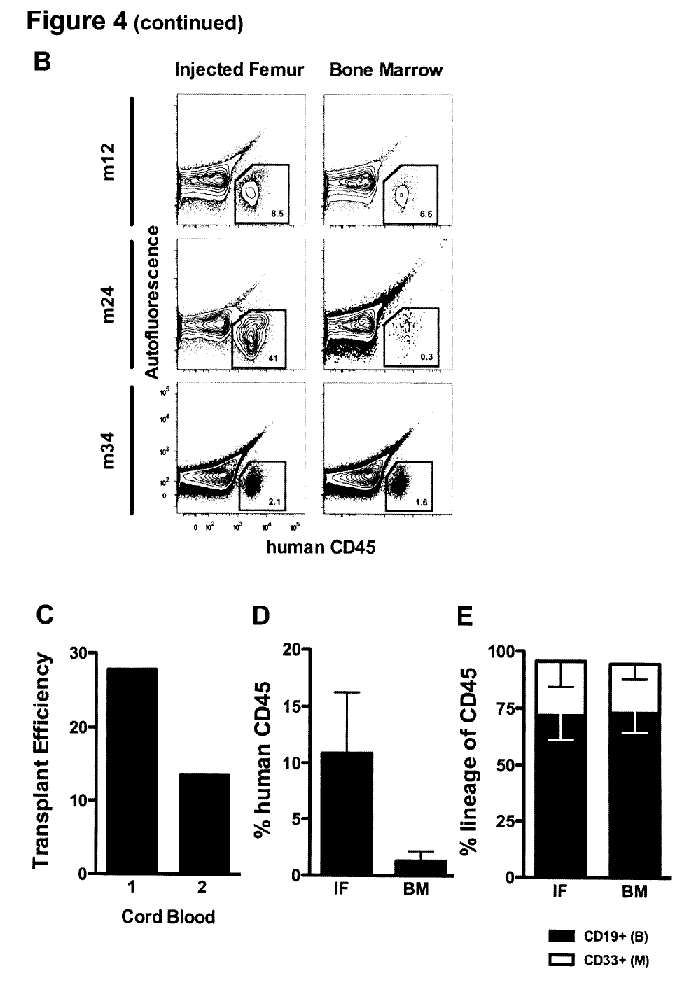

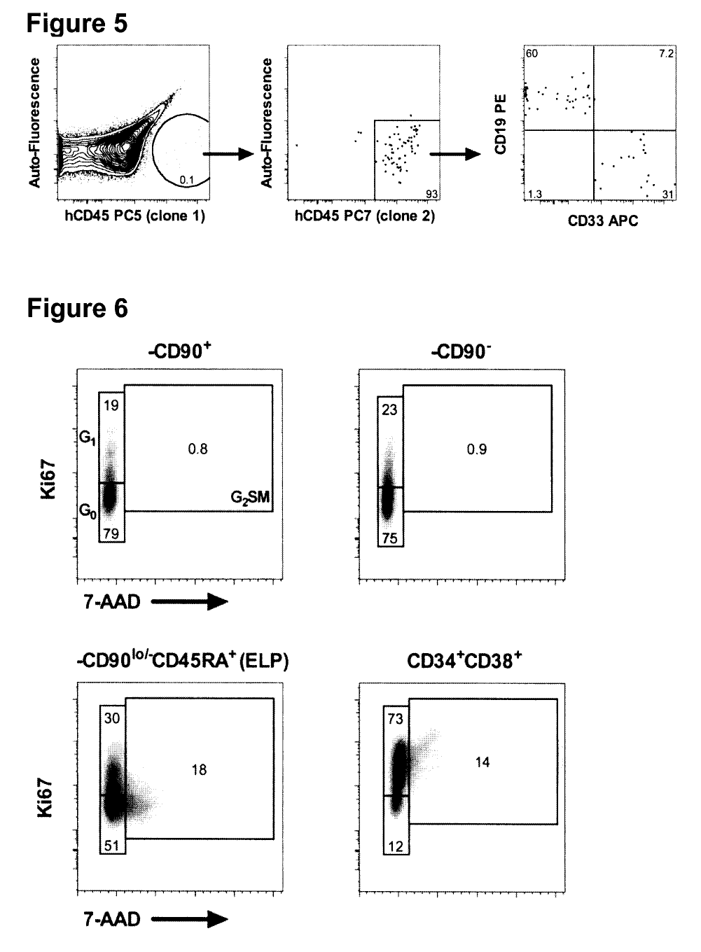

HSCs within the Thy1⁺ fraction, as shown with CD34⁺CD38⁻ cells. Indeed, Thy1⁺ Rho^{lo} cells showed a 2-fold enrichment for HSCs compared to Thy1⁺ alone. We next sought to determine if the addition of Rhodamine to Thy1⁺CD49f^{hi} cells would permit robust engraffinent of single human HSCs. We sorted single Thy1⁺Rho^{lo}CD49f^{hi} cells and transplanted them into NSG recipients (FIG. 4A). In our first experiment, 28% of recipients (5/18) transplanted with single Thy1⁺Rho^{lo}CD49f^{hi} cells displayed human chimerism 20 wk post transplant (FIG. 4B-C). Multilineage chimerism was observed in primary recipients and in 2 of 4 secondary mice despite only being transplanted with 20% of total marrow from primary recipients (data not shown). In a second experiment, we obtained a slightly lower frequency (14%, 3/22, FIG. 4C), although this cord blood had a dramatically lower level of Thy1 expression reflecting the intrinsic heterogeneity of primary human samples. Pooled analysis for levels of chimerism and multilineage differentiation potential of single Thy1⁺Rho^{lo}CD49f^{hi} cells is shown in FIG. 4D-E. The engraftment of single Thy1⁺Rho^{lo}CD49f^{hi} cells that displayed multilineage engraftment provide definitive experimental evidence that human HSCs express CD49f.

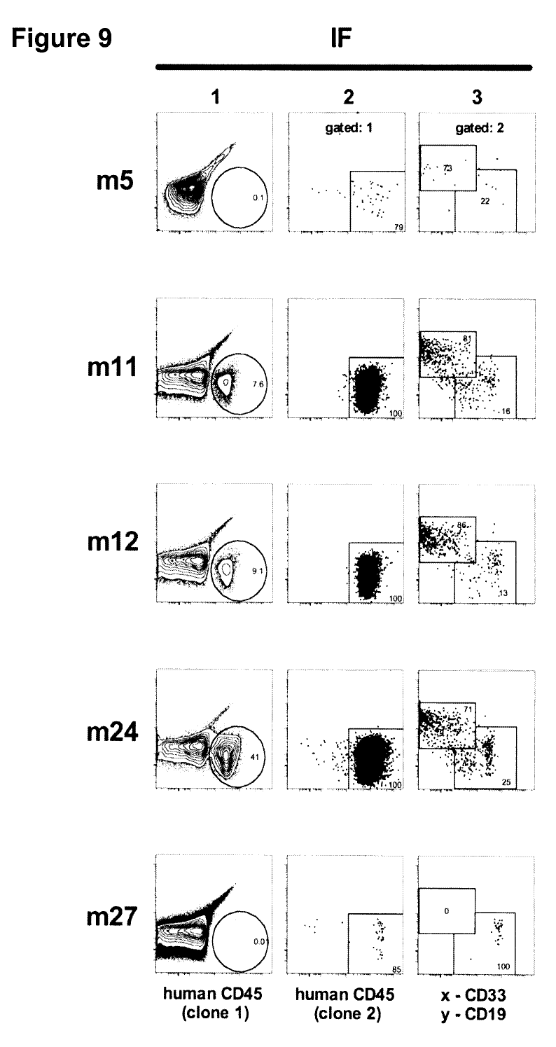

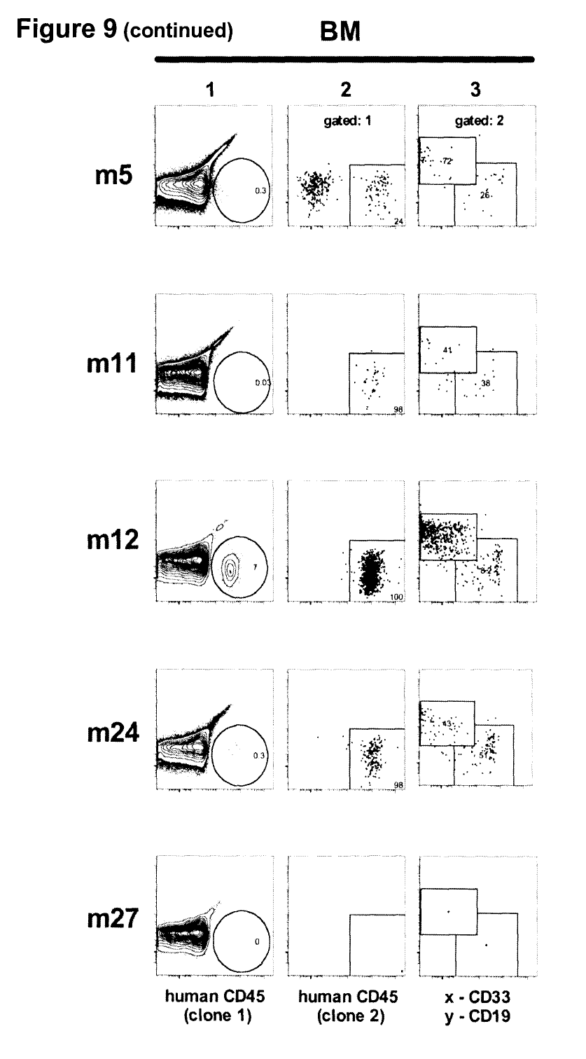

Applicants also conducted serial transplantation from primary recipients that received single cells as a second measure of self-renewal. Three of 17 mice transplanted with a dose equivalent of a single HSC, from CD90⁺ or CD90⁻ cells, engrafted secondary recipients. These data indicate that these cells can self-renew, but our ability to efficiently detect rare stem cell divisions is limited by the proportion of total bone marrow that was retransplanted (<20%) presenting a unique challenge to assessing clonal self-renewal events. Single HSCs injected into the femur must undergo self-renewal divisions to migrate to distant sites. In contrast, progenitors lack self-renewal capacity and are predicted to remain confined to the IF. As a proof of principle, Applicants injected sorted progenitors (early lymphoid precursor (ELP), common myeloid precursor (CMP) and granulocyte-macrophage precursor (GMP), and in each case human engraftment was observed in the IF, but not BM, SP or TH (FIG. 8 and data not shown). Therefore, in conjunction with long-term engraftment, the presence of a multilineage graft at non-injected sites is a sensitive surrogate test for self-renewal and migration of HSCs from the IF. In 4 of 5 mice engrafted from single CD90⁺Rho^{lo}CD49f^{hi} cells, human cells could be detected in the non-injected bones (FIG. 9) indicating that these cells give rise to long-term multilineage engraftment, self-renew and migrate, and thus represent bona fide human HSCs.

### Expression Profiling of Human HSC-Enriched Subsets

The ability to resolve single hematopoietic cells with long-term and multi-lineage capacity indicated that our CD49f positive subsets were highly enriched for human

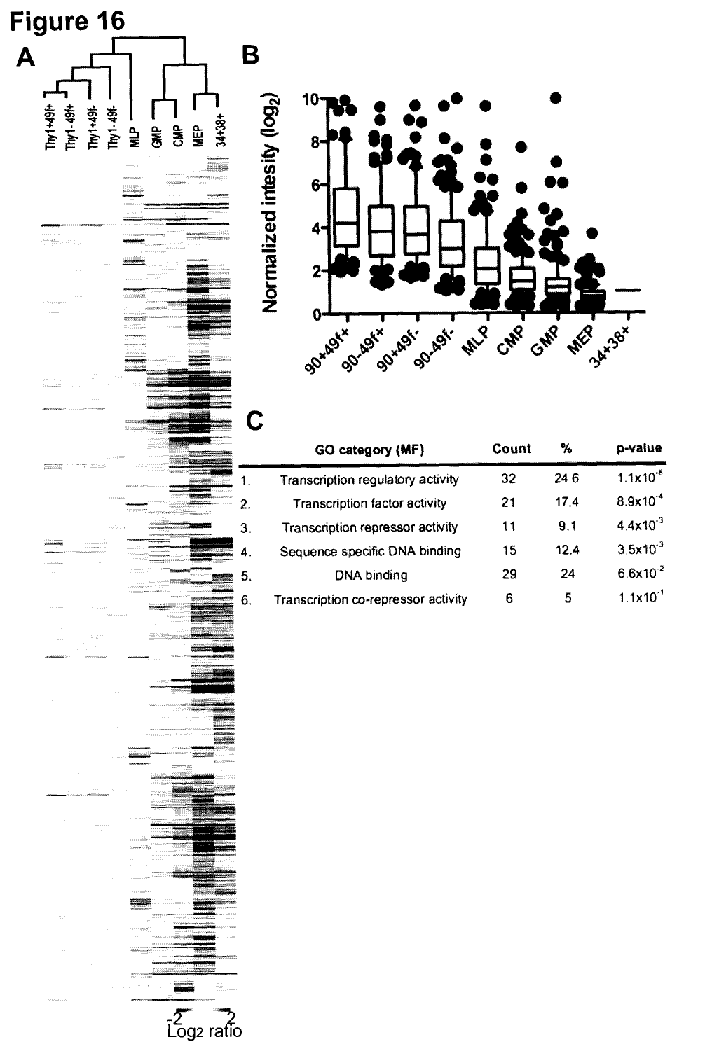

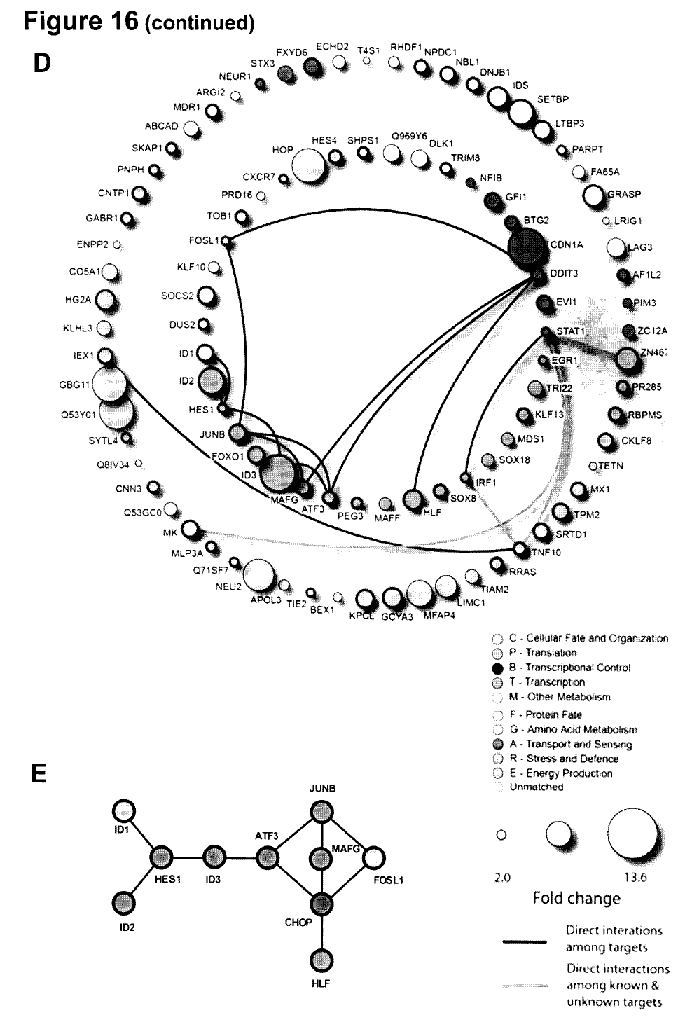

HSCs and presented an unprecedented opportunity to identify molecular regulators that govern its function. We performed gene expression analysis on both engrafting and non-engrafting CD49f subsets versus all major progenitor compartments recently identified by Doulatov et al. Unsupervised clustering revealed that the two HSC subsets (Thy1⁺CD49f^{hi} and Thy1⁻CD49f⁺) clustered together (FIG. 16A). Although small variations HSC frequency are observed between these fractions, no significant differences in gene expression were noted (10 genes at a 5% FDR). By contrast, the Thy1⁻CD49f⁻ fraction showed divergent gene expression from HSCs subsets, and clustered between HSCs and progenitors. Lineage committed progenitors, including MLP, CMP, GMP, MEP clustered separately in that respective order. This global expression pattern strongly supports our functional studies and substantiates the close functional similarity between Thy1⁺CD49f^{hi} and Thy1⁻CD49f⁺ HSCs while also revealing a distinct gene expression of Thy1⁻CD49f⁻ cells, and the gradual loss of self-renewal potential in progenitors.

To obtain a more precise view of the human HSC transcriptome, Applicants extracted the most significantly upregulated genes between the two HSC subsets versus non-engrafting fractions and all downstream progenitors. This analysis identified 146 genes whose expression was highest in HSCs and downregulated upon differentiation (FIG. 16B). A striking 17% of genes localized to two active chromosomal regions (1q21 and 6p21) and corresponded to core histone components (n=17) or MHC Class II genes (n=8). Gene ontology (GO) annotation by cellular function independently confirmed that nucleosome assembly (p=2.9×10⁻²⁰) and antigen processing (p=1.0×10⁻⁷) were the most enriched categories within this gene set. The biological significance of the active transcription of all classes of histones genes in largely quiescent HSCs is unclear. Upregulation of MHC Class II genes, including surface receptors such as CD74, support a role for human HSCs in immune-surveillance as recently shown in murine models whereby HSCs reside and can be stimulated by Toll-like receptor agonists in lymph tissues.

The remaining 121 genes were highly enriched for transcriptional regulators with several candidates previously implicated in HSC function (FIG. 16C, p=1.5×10⁻⁸). Remarkably, several notable candidates were represented amongst direct family members such as the inhibitor of DNA⁻binding (ID1, ID2, ID3), forkhead box protein (FOXO1, FOXN1), hairy and enhancer of split (HES1, HES4), homeobox (HOXB5, HOPX), sex determining region Y (SOX8, SOX18), tripartite motif-containing (TRIM22, TRIM8), kruppel-like factor (KLF10, KLF13), v-maf musculoaponeurotic fibrosarcoma oncogene (MAFF, MAFG) and ecotropic viral integration site (EVI1, EVI2B). The presence multiple members of a single gene family strongly implicates these genes in human HSC function. Other notable candidates that were actively transcribed in our HSC-enriched subsets included CDKN1A (p21), critical to the maintenance a quiescent stem cell state and PRDM16, that along with KLF10 (mentioned above) were recently identified in an in vivo gain-of-function RNAi screen as a novel regulator of murine HSC self-renewal. Overall, by limiting cellular heterogeneity from several contaminating hematopoietic subsets within CD34⁺CD38⁻ fraction, we reveal that several prominent stem cell regulators are expressed within the human HSC transcriptome.

While the above analysis highlighted critical genes implicated in stem cell function, 70% (84/121) of genes represented within this HSC-gene set no identifiable role in stem cells. We noted several genes within our HSC list that are expressed by human lymphocytes, primarily T-cells (ex. FAIM3, ENPP2, PNP, SKAP1, TNF10, CD83, TOB1, ATF3). Interestingly, GO annotation of this specific 84 gene-set revealed significant enrichment for regulators of T-cell differentiation (p=8.3×10⁻³) and immune response (p=7.4×10⁻²). In particular, transducer of ERBB2 (TOB1) is a master regulator of T-cell quiescence and essential for the long-term survival of peripheral T-cells. Interestingly, both members of the TOB gene family (TOB1 and BTG2) are present within this gene set. In murine hematopoiesis, long-lived lymphocytes such as memory B- and T-cells share a significant number of transcripts expressed by long-term HSCs linking the self-renewal phenotype shared by these divergent cell types at the molecular level.

### Transcriptional Networks Within HSC-Enriched Subsets

Biological processes are predicated on the cooperation of genes that are organized into pathways and networks that provide the basis for virtually all cellular functions. To determine if any genetic interactions exist with of HSC gene set, we developed a connectivity map, a strategy commonly utilized to interrogate gene expression data set. To reduce complexity, only transcription factors and genes with associated stem cell function were segregated from those with no known function (FIG. 16D). Remarkably, this analysis revealed 25% (10/42) of genes collapsed on a single transcriptional network with two predicted functional molecules. The first module converged on a single node, HES1, that directly interacted with all members of the ID gene family (ID1, 1D2, ID3) (FIG. 16E). Importantly, these interactions unite and implicate Notch and BMP signaling pathways in human HSC function. Both functional modules were bridged by 1D3, a highly expressed gene in human HSCs, linked through a direct interaction with AFT3. The second module was highly integrated and consisted of AFT3, CHOP, FOSL1, JUNB, MAFG and HLF (FIG. 16E) Several members within this network have been implicated in stem cell function. In particular, overexpression of HES1 and HLF in Lin⁻CB increased engraftment potential in Nod-scid mice. In general, complex cellular functions such as self-renewal are likely governed a collaboration of a large number of genes. Our predicted HSC connectivity map, albeit transcriptionally based, which include several prominent stem cell regulators provides further validity that HSC-gene list accurately reveals the human HSC transcription.

### Absence of CD49f Expression on Human Multi-Potent Progenitors

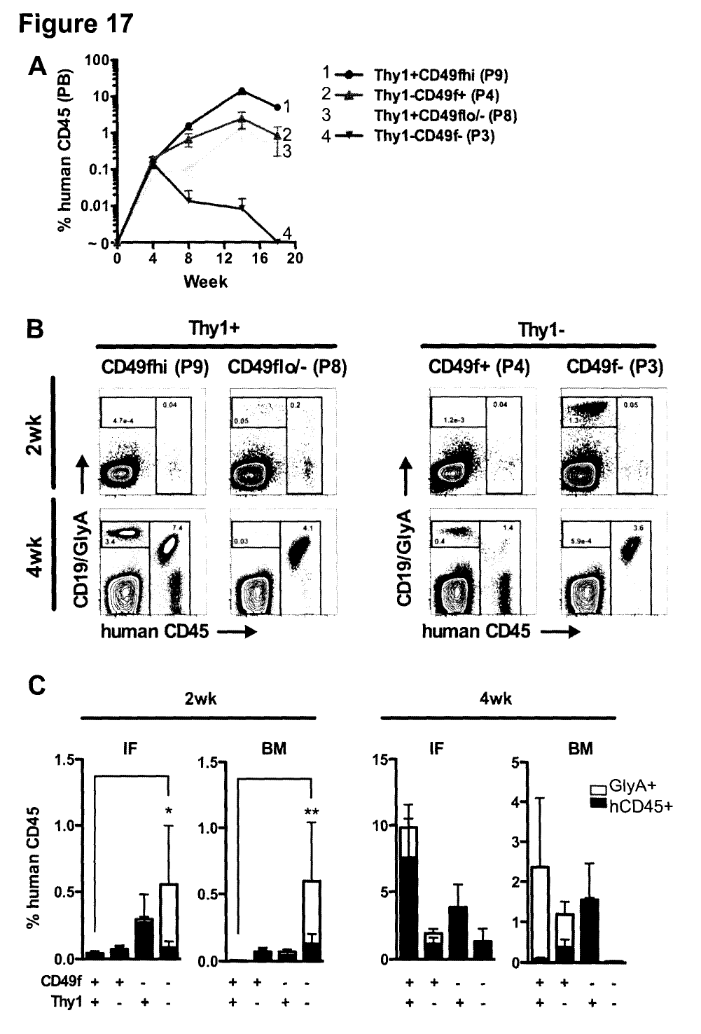

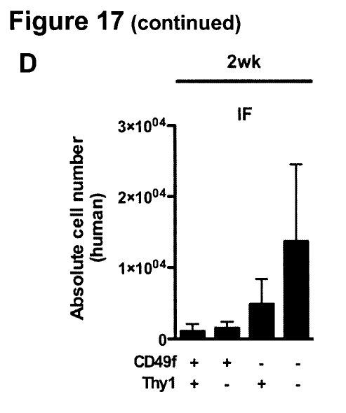

The Thy1⁻ fraction of neonatal cord blood has been proposed to represent MPPs despite retaining the capacity to engraft secondary animals as we and others have shown. The ability to prospectively segregate HSCs within Thy1⁻ cells using CD49f expression provides conclusive evidence that this fraction is heterogeneous. And therefore, definitive identification of human MPPs still awaits. Our engraftment studies indicate that Thy1⁻CD49f⁻ cells lack the ability to engraft long-term and display a divergent gene expression program when compared to CD49f positive HSC subsets (FIG. 17A). Along with the observation that the majority of Thy1⁻ cells do not express CD49f, we hypothesized that human MPPs are demarcated by loss of CD49f expression. We performed kinetic analysis and monitored the peripheral blood and marrow of NSG recipients transplanted with all four Thy1 and CD49f subsets over 20 wk. While peripheral blood chimerism in recipients of HSC-enriched fractions gradually increased over time (Thy1⁺CD49f^{hi} and Thy1⁻CD49f⁺), engraftment of Thy1⁻CD49f⁻ cells peaked between 2-4 wk and slowly decreased thereafter (FIG. 17A). The bone marrow of Thy1⁻ CD49f⁻ recipients displayed significantly higher levels of engraftment compared to other fractions at 2 wks in the both in the injected femur and non-injected bones indicating that these cells have a higher differentiation capacity then HSCs immediately after transplant (FIG. 19B-D). Erythroid cells, B cells, monocytes and granulocytes were generated in the bone marrow of Thy1⁻CD49f⁻ mice (FIG. 17B). HSC-enriched fractions displayed a delay in engraftment kinetics, however by 4 wks all lineages including B-lymphoid, erythroid and other myeloid cells were present (FIG. 17B). Engraftment kinetics of Thy1⁺CD49f^{lo/-} cells were intermediate to HSC and Thy1⁻ CD49f⁻ subsets (FIG. 17A-D). These data demonstrate that Thy1⁻CD49f⁻ cells retain the capacity to differentiate into major hematopoietic lineages, but lack the capacity to engraft long-term indicating that these are bona fide MPPs. Critically, MPPs generated a robust erythroid output in vivo and in colony assays (data not shown) suggesting that human HSCs do not necessarily undergo an erythroid-MK restriction proposed to be the earliest lineage decision in mouse hematopoiesis.

The inability of Thy1⁻CD49f⁻ cells to sustain long-term engraftment indicate that these cells have limited capacity to self-renew. During fate specification, transcription factors that are associated with a particular cell lineage are upregulated to suppress self-renewal program in HSCs. To support our functional analysis, we investigated whether genes upregualted in Thy1⁻CD49f⁻ cells compared to HSC subsets. This analysis identified 86 genes enriched in transcriptional regulatory activity (p=8.4×10⁻²), and included several genes linked with lymphoid and myeloid lineage priming and negative control of self-renewal, including IKZF1, PLZF, SMAD3 and MYC. In particular, Myc expression in murine HSCs leads to loss of self-renewal activity at the expense of differentiation by represses N-cadherin and integrins molecules, including CD49f, providing a mechanistic basis of loss of CD49f expression on human MPPs. Additionally, there was also an induction of DNA damage response transcripts (GADD45G, XRCC2, CDKN1B, etc) consistent with both reduced DNA repair capacity of HSCs and increased preparedness in anticipation of lymphoid gene rearrangement to follow. The gene expression program in Thy1⁻CD49f⁻ cells supports our functional analysis in NSG mice and strongly suggests that loss of CD49f expression is required to identify human MPPs.

### Discussion

Applicants resolve the extent of stem cell heterogeneity within CD34⁺CD38⁻ fraction of neonatal cord blood and reveal the existence of multiple distinct cellular subsets that vary in their capacity to engraft NSG mice. Although widely accepted to be highly enriched for human HSCs, our results clearly indicate that this fraction remains dramatically heterogenous. HSCs within this fraction constitute the rarest functional cell type and reside amongst more abundant MLPs and MPPs. We found that HSCs within this compartment can be enriched according to high levels of Thy1 expression, although the discovery of CD49f as a novel marker of human HSCs was critical in providing absolute resolution. Remarkably, this resolution permitted the detection of single hematopoietic cells endowed with extensive self-renewal and long-term engraftment capacity and represents the first definitive identification of human HSCs. Together with Doulatov et al., these studies communicate a comprehensive roadmap of the major cellular classes that comprise the human blood system.

Over a decade has elapsed since human HSCs were shown to be classified according to Thy1 or CD38 expression. Extensive xenografting has clearly indicated that human HSCs were minor constituents within these fractions and that the identification of additional markers was urgently required to advance the field. The present data establishes that virtually all human HSCs express CD49f and demonstrate that HSCs reside within both Thy1⁺ and Thy1⁻ subfractions of CD34⁺CD38⁻CD45RA⁻ cells. The presence of HSCs within Thy1⁻ cells was unexpected as loss of Thy1 expression is widely considered to denote HSC differentiation; this raises the debate of whether both Thy1 and CD49f are required to identify human HSCs? Since the bulk of Thy1⁺ cells versus a minority of Thy1⁻ cells express CD49f, we conclude that the inclusion of both markers are required to yield the highest proportion of HSCs. Our ability to efficiently resolve human HSCs at single cell resolution in NSG mice is a testament to this fact. Although extensive LD analysis provide ample evidence that CD49f can dramatically enrich for human HSCs over current standards, we believe that further ‘humanization’ of xenograft models will likely reveal a higher estimate. Additionally, assessment of the functional role of CD49f on human HSCs is also warranted. High expression levels of CD49f on both normal and malignant human stem cells from other tissue types do suggest a widespread and conserved role for this integrin in its ability to anchor human stem cells within their niche.

Changes in cellular adhesion requirements, such as the ability to anchor against a basement membrane, during hematopoietic cell specification can potentially reconcile the absence of CD49f expression on human MPPs. Majeti et al. were the first to propose that human MPPs are Thy1⁻, however inclusion of CD49f is required for absolute delineation. Unsupervised cluster analysis and increased expression of genes related to lineage priming support its identification. By restricting our analysis to genes highly expressed within our HSC-subsets we revealed several notable transcription factors implicated self-renewal, although a significant proportion of genes remain unannotated with respect to stem cell function.

The identification of genes whose transcription is restricted to HSCs is the first step towards decoding the molecular networks that control stem cell function.

### Example 2

### Identification of an Early Lymphoid Progenitor with Monocytic Potential

### Methods

Sample collection and sorting. CB samples were obtained according to the procedures approved by the institutional review boards of the University Health Network and Trillium Hospital. Lineage-depleted (Lin⁻) CB cells were purified by negative selection using the StemSep® Human Progenitor Cell Enrichment Kit according to the manufacturer's protocol (StemCell Technologies). CD34⁺-selected BM and mPB cells were obtained from Lonza. Lin⁻ cells were thawed and stained at 1×10⁶ cells/100 μl with CD45RA FITC (4 μl), CD135 PE (8 μl), CD7 PE-Cy5 (Coulter; 2 μl), CD10 APC (4 CD38 PE-Cy7 (3 μl), CD34 APC-Cy7 (4 μl), and CD90 Biotin (4 μl) (+Qdot 605 2°; 2 μl). Cells were flow sorted (1-8 cells/well, in single cell or limiting dilution format) directly into 96-well plates pre-seeded with stroma by a single cell deposition unit coupled to BD FACSAria sorter, providing the indicated number of cells in 88% of wells, as assessed by counting the number of cells deposited into empty wells after single cell sorting. The purity of single cell sorting was routinely assessed by recovering sorted cells and found to be >99%. All antibodies from BD, unless stated.

Clonal assays on stroma. MS-5 stroma was seeded in 96-well plates (Nunc) coated with 0.2% gelatin at 5×10³ cells/well in H5100 media (StemCell Technologies) plus cytokines (in ng/ml): SCF (100), IL-7 (20), TPO (50), IL-2 (10), and in some experiments: GM-CSF (20), G-CSF (20), and M-CSF (10). All cytokines from R&D. After 24-48 hrs, single sorted progenitor cells were sorted onto stromal monolayers. For co-culture experiments, MS-5 and MS-5/DL4 were mixed at 4:1 ratio and cultured with SCF, IL-7, TPO, FLT3 (10), and GM-CSF. MS-5 cultures were maintained for 4 wks with weekly ½ media changes. Wells were resuspended by physical dissociation, filtered through Nytex membrane, stained with: CD45, CD19, CD14, CD15, CD33, CD56, CD33, and analyzed by high-throughput flow cytometry. DL4 co-cultures were analyzed with CD5, CD7, CD33, CD11b and CD19. OP9 stroma was seeded in 96-well plates (Nunc) at 5×10³ cells/well in aMEM (Gibco) with 20% FBS. Sorted progenitors were expanded for 9 days with SCF (100), TPO (50), IL-7 (10), FLT3 (10), then differentiated into DCs with GM-CSF (50) and IL-4 (20), or macrophages with M-CSF (20) and IL-6 (20), or a combination of these cytokines, for 7 days. OP9-DL1 stroma was seeded in 96-well plates at 5×10³ cells/well in aMEM (Gibco), 20% FBS (previously tested for T-cell support), plus FLT-3 (5) and IL-7 (5). Cells were transferred onto fresh stroma 2×a week, or as needed and analyzed for T-cell proliferation after 7-8 wks with CD45, CD3, CD5, CD7, CD4, CD8. Clones were required to have >20 CD45⁺ gated events (of indicated cell-surface phenotypes) to be scored as positive. MC cultures were prepared as described¹⁶.

Quantitative PCR. RNA was extracted from ˜2×10⁴ sorted progenitors using Trizol® reagent (Invitrogen), DNAse I-treated, and reverse transcribed with SuperScript™ II (Invitrogen). Real-time PCR reactions were prepared using the SYBR® Green PCR Master Mix (Applied Biosystems), 200 nM primers (Qiagen), and >20 ng cDNA. Reactions were performed in triplicate on Applied Biosystems 7900HT. Gene expression was quantified using the SDS software (Applied Biosystems) based on the standard curve method.

Microarray analysis. Total RNA extracted from 5-10×10³ cells from HSC, MLP, CMP, GMP and MEP populations (Table 1) using Trizol® (Invitrogen) was amplified, hybridized to Illumina HT-12 microarrays, and analyzed using GeneSpring GX 10.0.2 software (Agilent Technologies) after quantile normalization. Differentially expressed probes were determined using ANOVA analysis followed by Benjamini Hochberg FDR correction (0.05). MLP-specific gene expression signature was generated from probes showing MLP>MEP expression pattern, after an initial filter for probes differentially expressed at least 2-fold between any two populations, except between HSC and MPP. Cluster analysis was performed with MeV.

Mouse transplantation. NOD/LtSz-scidIL2Re^{null} (NSG) (Jackson Laboratory) were bred and housed at the TMDT/UHN animal care facility. Animal experiments were performed in accordance to institutional guidelines approved by UHN Animal care committee. Mice were sublethally irradiated (200-250 cGy) 24 h before transplant. Cells were transplanted intrafemorally into anesthetized mice, as previously described. Briefly, a 27 g needle was used to drill the right femur, and cells were transplanted in a 25 μL volume using an 28.5 g insulin needle. Mice were sacrificed after 2 and 4 wks for progenitor, or 10 wks for HSC, analysis. Marrow was isolated by flushing bone cavities with 2 mL IMDM, and 100 μL. stained for surface markers: CD45, CD19, CD33, CD14, CD15, CD56. For analysis of HSC-derived hierarchy, human progenitors were isolated from pooled bone marrow using the Mouse/Human Chimera Enrichment Kit (StemCell Technologies) according to the manufacturer's protocol, with the addition of 100 μL/mL StemSep Human Hematopoietic Progenitor Enrichment Cocktail (StemCell Technologies) and the anti-biotin antibody.

Dendritic cell cultures. OP9 stroma was seeded in 6-well plates at 1×10⁶ cells/well in αMEM, 20% FBS, plus SCF (100), FLT-3 (100), TPO (50), and IL-7 (20). Human progenitors were sorted from CB, BM or mPB and seeded on OP9 stroma at 100-1,000 cells/well. Cultures were carried for 2 wks, with bi-weekly ½ media change. Wells were resuspended by physical dissociation, Nytex-filtered, and CD45⁺ cells sorted into suspension cultures with aMEM, 20% FBS, plus GM-CSF (50) and IL-4 (20). Cultures were carried for 5 d with 1×media change. Cells were harvested and 2×10⁵ cells/well matured in RPMI, 2% human serum, L-glutamine, plus TLR ligands for a total of 24 hrs. IFN/LPS: IFNγ (1000 U) 4 h, LPS (10) 20 h; LPS (10); TNF/IL1β: TNFα (10), IL-1β (10), IL-6 (1000 IU), PGE2 (10 μM); poly I:C (10,000); CpG (10 μM); Imiquimod (1,000); LTA (1,000); IFN/LTA: IFNγ (1000 U) 4 h, LTA (1,000) 20 h. Cells were stained with CD14, CD80, CD86, CD83, CD40 or CD14, HLA-DR, CD11c, CD1a, CD11b and analyzed by FACS; all antibodies from BD. Cytokine secretion was measured by ELISA as described.

Statistics. Clonal data is based on single cell or limiting dilution experiments. For single cell experiments, clonogenic efficiency is reported as % positive wells. Limiting dilution data is represented as the estimated limiting dilution frequency ±95% confidence interval. Limiting dilution analysis was performed using the online software provided by WEHI bioinformatics (http://bioinf.wehi.edu.au/software/elda/index.html, Hu Y. and Smyth G. (2009), ELDA: Limiting dilution analysis for comparing depleted and enriched populations, Walter and Eliza Hall Institute of Medical Research, Australia).

### Results

### Clonal Assays of Human Hematopoiesis

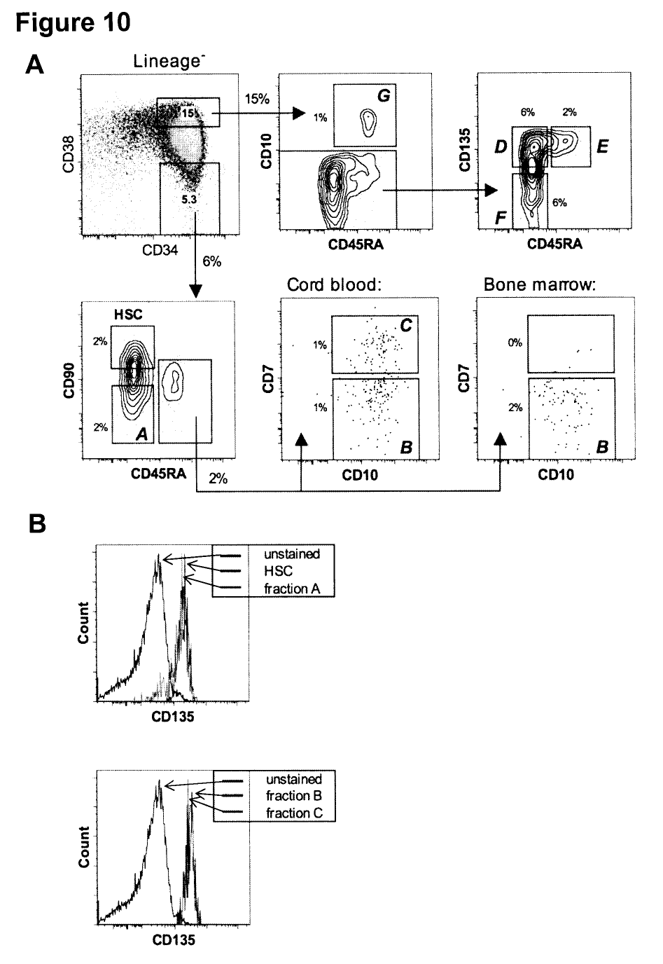

To investigate the composition of the human progenitor hierarchy, we used flow sorting to isolate progenitor (CD34⁺) fractions based on the expression of CD45RA, CD135 (FLT3), CD7, CD10, CD38 and CD90 (Thy1). Our studies established that this combination provides a meaningful separation of human progenitors into functionally distinct subsets. Because age-related developmental changes may affect the composition of the progenitor compartment, we isolated progenitors from neonatal CB, which contains a mixture of fetal and adult cells, as well as adult BM. Staining of lineage-depleted (Lin⁻) or CD34⁺-selected samples with this marker panel revealed 7 distinct progenitor fractions (labeled fractions A-G) in addition to CD34⁺CD38⁻ Thy1⁺CD45RA⁻ HSCs (FIG. 10 and Table 1). These populations could also be resolved in unfractionated BM or CB making this panel more suitable for smaller samples or diagnostic applications.

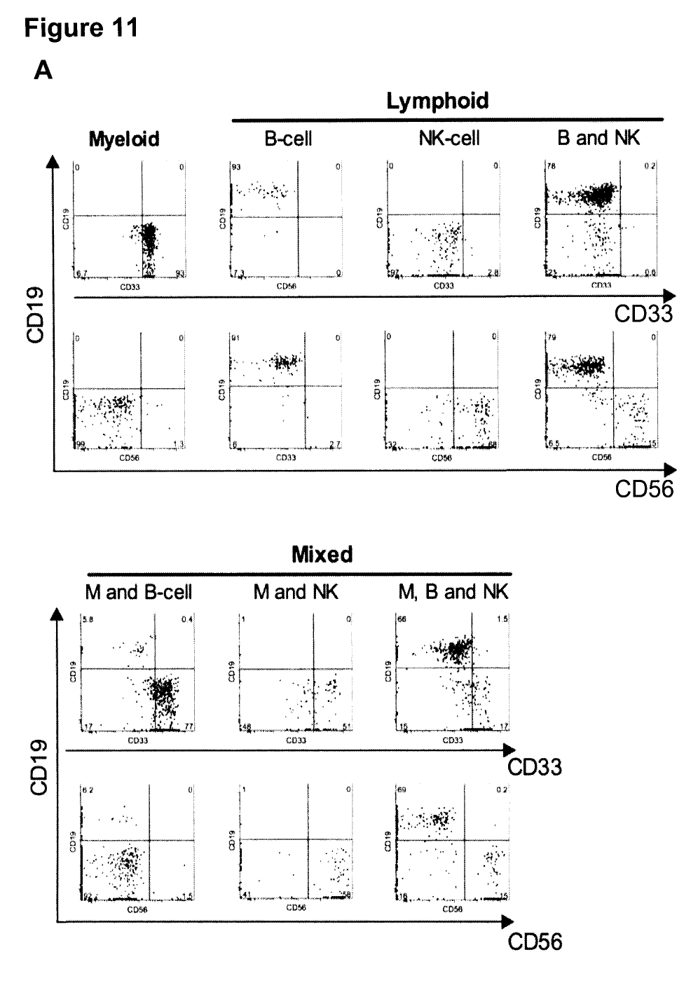

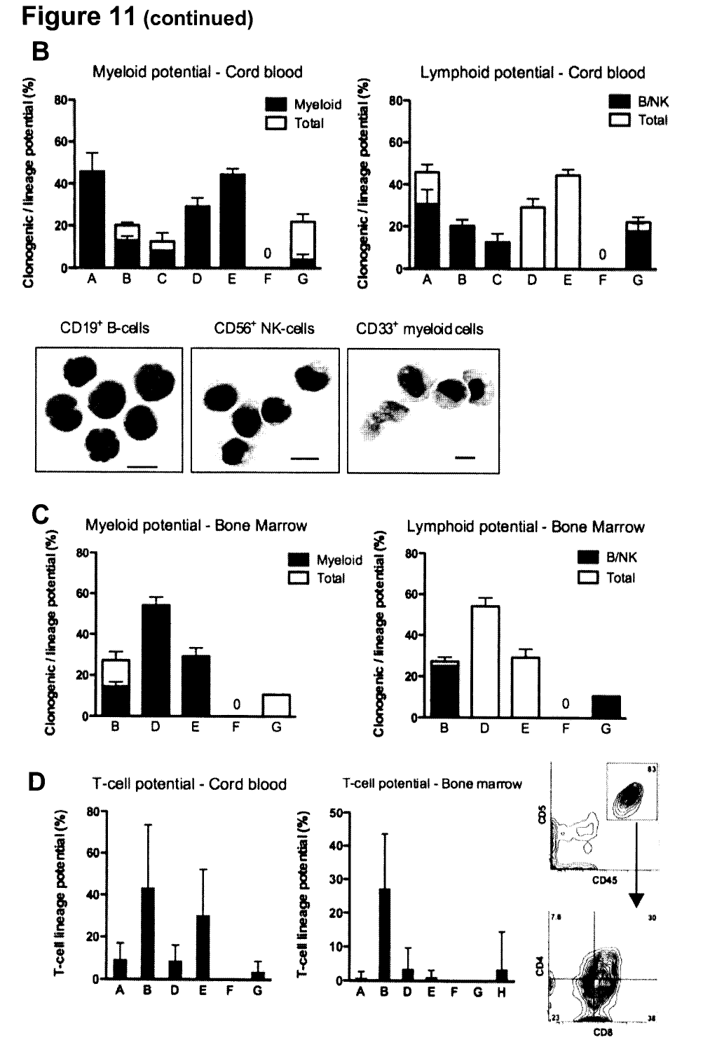

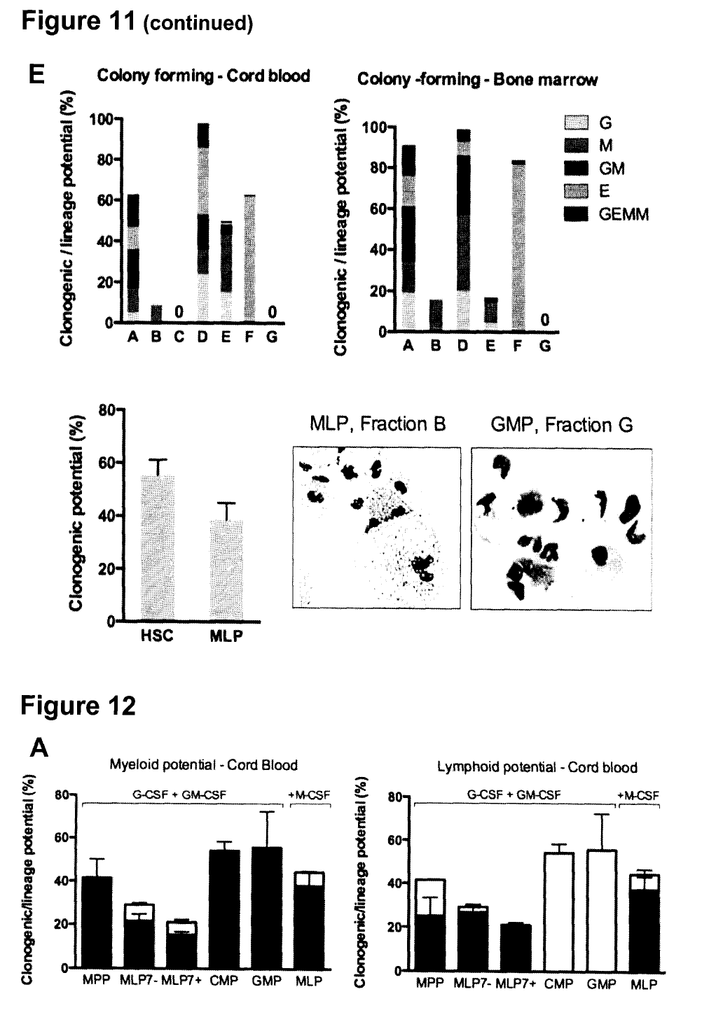

The shortcomings of previous approaches were in part due to the lack of assay to efficiently detect lymphoid and myeloid lineages from single human cells. Murine MS-5 stromal cells support the development of human myeloid, B cell, NK and mixed lympho-myeloid colonies in the presence of stem cell factor (SCF), thrombopoietin (TPO), interleukin-7 (IL-7) and IL-2²¹. Single cord blood CD34⁺CD38⁻Thy1⁻CD45RA⁻ cells proposed to be human multi-potent progenitors (MPPs)²², seeded in these conditions gave rise to all 7 possible colony types with a high cloning efficiency (FIG. 11A, fraction A, 45% cloning efficiency). In addition, we employed OP9-DL1 stromal assays to detect T cell potential²³, and conventional colony (CFU) assays for myeloid and erythroid lineages. Of note, MPPs displayed reduced efficiency in OP9-DL1 assays likely owing to Notch-mediated inhibition of differentiation^{24, 25}, but had T cell potential in vivo (Notta et al., manuscript in preparation). Evidence of lineage fate potential of any purified population is definitive only when assessment is done at the level of single cells. Thus, we used limiting dilution analysis or deposition of single cells, which resulted in similar estimates of clonogenic potential (FIG. 11B) providing the basis for a precise clonal read-out of lineage potential.

### Human Myeloid Progenitors

In our analysis of lineage potential on MS-5 stroma, progenitor fractions D and E (Table 1) gave rise exclusively to myeloid, but not B cell or NK colonies (FIG. 11B,C, with cloning efficiency ranging from 54% (fraction D, BM), 44% (E, CB) to 29% (D, CB and E, BM). With the exception of fraction E from CB, these cells had no T cell potential (FIG. 11D). Both D and E fractions gave rise to myeloid colonies in CFC assays, and D also generated erythroid and myelo-erythroid colonies consistent with a common progenitor of myeloid lineages (CMP; FIG. 11E). By contrast, erythroid colonies were never observed from fraction E cells, consistent with a more restricted progenitor of granulocyte and monocyte lineages (GMP; FIG. 11E). It is unclear why GMPs in CB had significant T cell potential and will be the subject of future investigation, however a similar finding has been reported recently in the mouse²⁶. CMPs from CB, but not BM, possessed serial replating potential, albeit with a lower capacity than multipotent cells. In contrast to the Flt3⁺ fractions, fraction F cells produced no colonies in the MS-5 or OP9-DL1 assays (FIG. 11B-D), but gave rise to erythroid colonies in CFU assays, with no detectable myeloid potential, consistent with a restricted E-MK progenitor (MEP; FIG. 11E). These results establish the identity of key myeloid progenitor types from both neonatal and adult sources and indicate that myeloid commitment in human hematopoiesis proceeds along a developmental path consistent with the classical model.

### Human Multi-Lymphoid Progenitors

Previous reports of human MLPs with B, T and NK cell potential placed them in the CD10⁺CD24⁻ or the CD38⁻CD7⁺ fractions^{17, 19}. To refine this analysis, we determined the lineage potential of progenitor fractions expressing lymphoid markers CD7 or CD10. CD10 was expressed by a subset of CD34⁺CD38⁺ cells (fraction G) and a distinct fraction of Thy1^{-/lo}CD45RA⁺ cells within the CD34⁺CD38⁻ stem cell compartment (FIG. 10 and Table 1). Fraction G cells gave rise to B and NK colonies on MS-5 stroma, with a bias for NK lineage, and lacked appreciable myeloid potential in CFU assays (FIG. 11B, C; cloning efficiency=24% CB, 13% BM). This fraction had no detectable T cell potential in OP9-DL1 assays (FIG. 11D) indicating that these cells were precursors of B and NK cells (pre-B-NK), but not MLPs.

We next tested the developmental potential of Thy1^{-/lo}CD45RA⁺ cells within the CD34⁺CD38⁻ compartment. In CB, these cells expressed CD10 and could be sub-divided into CD7⁻ (fraction B) and CD7⁺ (fraction C) populations; by contrast, BM cells were uniformly CD7⁻ (FIG. 10). These cells comprised 1-2% of Lin⁻ CB, and their frequency was unchanged in adult BM. In limiting dilution and single cell plating on MS-5 stroma, every colony generated by fraction B cells from CB contained lymphoid (B, NK, or B-NK) cells, and 57% of colonies also contained CD33⁺CD111b⁺ myeloid cells (FIG. 11B and Table 2; cloning efficiency=19%). However, these progenitors never produced myeloid colonies without lymphoid progeny. Similar results were obtained with fraction B cells isolated from BM (FIG. 11C and Table 2; cloning efficiency=27%), with no differences in myeloid, B-, or NK-lineage outputs between neonatal and adult samples (Table 2). Fraction B cells displayed robust T cell potential on OP9-DL1 stroma (FIG. 11D), with higher cloning efficiency and proliferative potential from CB compared to adult cells, consistent with the diminished output of T lymphocytes with aging²⁷ (FIG. 11D; cloning efficiency=45% CB, 27% BM). Thus, these progenitors could be identified as MLPs that were not restricted to the lymphoid lineages, and hence they could not be defined as CLPs, which are expected to be lymphoid-restricted.

To assess the myeloid potential of human MLPs we used CFU assays. CB and BM MLPs gave rise to macrophage CFU-M, independently established on the basis of their CD14⁺CD11b⁺ phenotype and cell morphology (FIG. 11E). No granulocytic CFU-G colonies arose from MLPs. Since GMPs always gave rise to a mixture of CFU-G and CFU-M under the same conditions (FIG. 11E), we can conclude that MLPs retain only macrophage potential. While only 10% of freshly sorted CB MLPs formed colonies, CFU efficiency could be dramatically increased by pre-culturing them on OP9 stroma. After 4 d of OP9 pre-culture, 50% of MLPs generated CFU-M colonies, comparable to Thy1⁺ HSCs (FIG. 11E, right panel). MLP-derived colonies could not be replated indicating that MLPs do not possess self-renewal capacity. Thus, single MLPs could give rise to B, T, NK cells and macrophages, but lacked granulocytic or erythroid lineage potential.

We next tested the developmental potential of the CD7⁺ cells within the CD34⁺CD38⁻ Thy1^{-/lo}CD45RA⁺ compartment (fraction C) that were previously proposed to be CLPs in CB¹⁹ (not found in BM, FIG. 10). Surprisingly, their lineage output was identical to the CD7⁻ MLPs, albeit at a lower cloning efficiency, with a similar proportion of lymphoid and lympho-myeloid colonies (FIG. 11B and Table 2; cloning efficiency=11%). Fraction C MLPs did not form colonies in CFU assays (FIG. 11E) indicating that the standard colony assays may underestimate myeloid potential and providing an explanation as to why it was not detected in prior reports¹⁹. Thus, CD34⁺CD38⁻ Thy1^{-lo}CD45RA⁺ cells are MLPs irrespective of their CD7 expression.

MLPs Differentiate into B, NK Cells and Monocytes

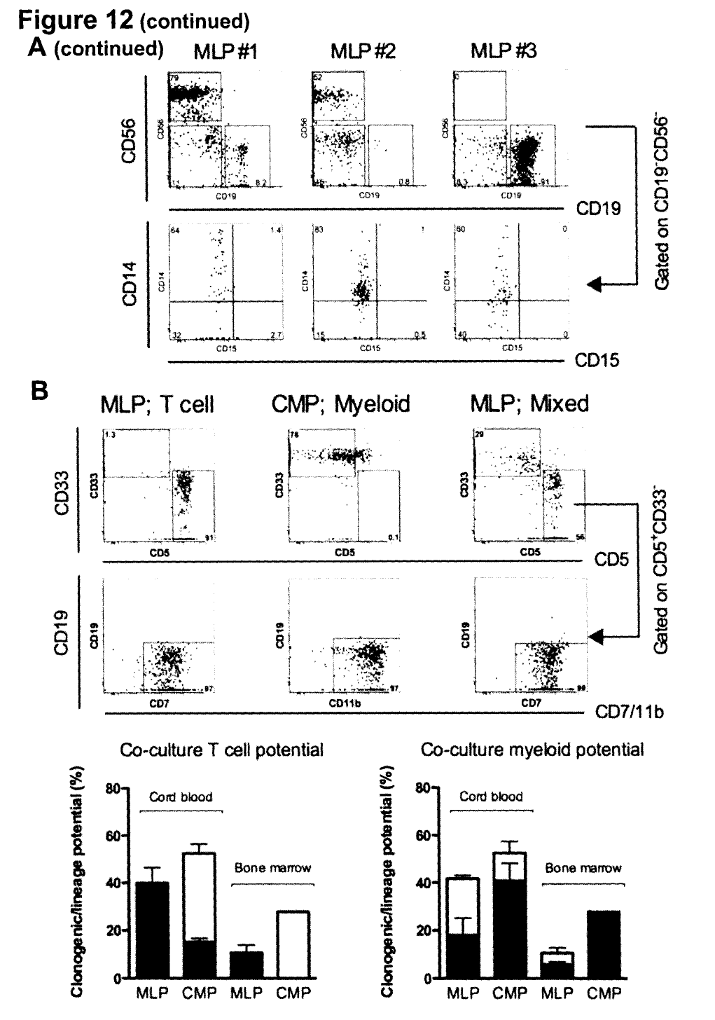

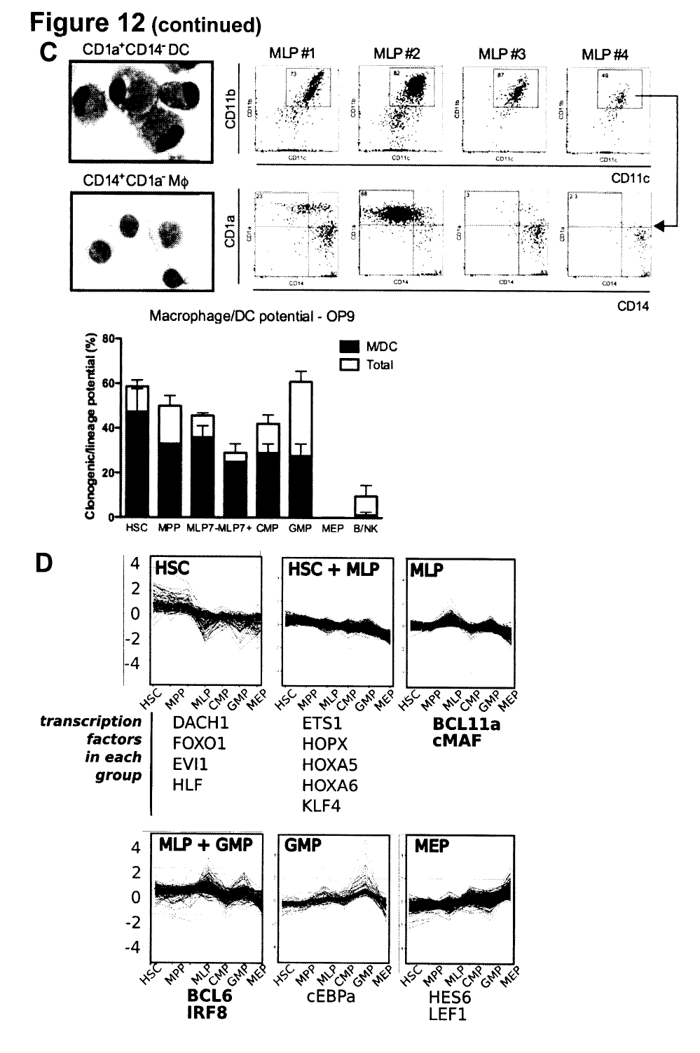

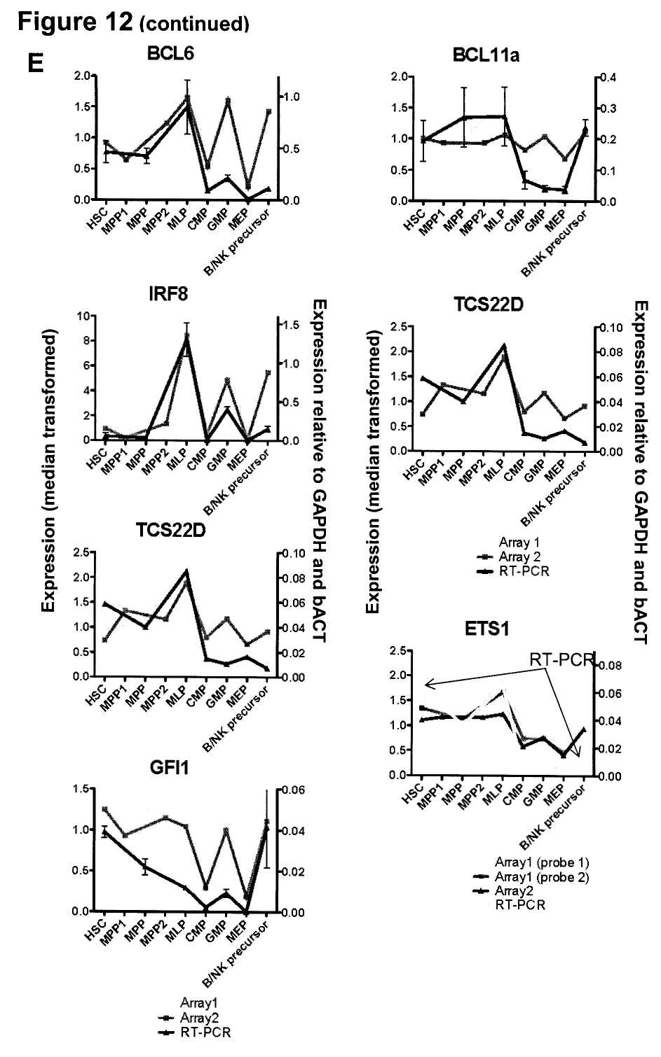

We undertook a more rigorous analysis of human MLPs to confirm their myeloid potential. The fact that only half of MLP colonies exhibited bi-potent myelo-lymphoid potential could be due to inadequate myeloid support in our standard MS-5 assays. To improve detection of myeloid maturation, we cultured single MLPs on MS-5 in the presence of myeloid cytokines, granulocyte colony-stimulating factor (G-CSF) and granulocyte macrophage colony-stimulating factor (GM-CSF). Clonal efficiency was improved under these conditions, with 21% of CD7⁺ and 29% of CD7⁻ CB MLPs giving rise to colonies (FIG. 3A12A). Inclusion of a monocytic cytokine, macrophage colony-stimulating factor (M-CSF), further augmented cloning efficiency to 44% (FIG. 12A). However, taking into account the 77% detection efficiency of the single cell sorting protocol these data suggest that 57% of successfully seeded MLPs have myeloid potential. B or NK cells were present in nearly all positive wells indicating that myeloid cytokines did not exert instructive effects on lymphoid commitment of CB MLPs. Notably, 85% of positive wells with B or NK lymphocytes also contained CD14⁺CD11b⁺ monocytes or macrophages, conclusively demonstrating that MLPs have the capacity to give rise to both lymphoid and monocytic lineages (FIG. 12A). Of interest, exposure of BM MLPs to myeloid cytokines instructed myelo-monocytic outcome demonstrating that cytokine signals are interpreted differently by neonatal and adult MLPs. None of the fractions we characterized had lineage potential consistent with a CLP; rather all progenitors with multi-lymphoid output also retained macrophage lineage potential.

MLPs Differentiate into T and Myeloid Cells

Due to the inability to read-out T cell potential in the same assay as the other lineages, we could not rule out the possibility that T cells are produced from a different precursor in the MLP fraction. To address this possibility, we developed a co-culture system in which MS-5 transduced with the Delta-like 4 gene were cultured with untransduced MS-5 cells enabling T lymphoid and myeloid development in a single well. Single MLPs isolated from CB or BM gave rise to CD7⁺CD5⁺ CD19⁻ T cell and mixed T cell-CD33⁺CD11b⁺ myeloid, but not myeloid-only, colonies (FIG. 12B). By contrast, CMPs from CB or BM generated only myeloid colonies under the same conditions (FIG. 12B). These data confirm that MLPs can give rise both T lymphoid and myeloid lineages.

MLPs Differentiate into Macrophages and DCs

Dendritic cells (DCs) are potent antigen-presenting cells that share a common progenitor with macrophages (the macrophage-DC progenitor, or MDP)²⁸⁻³⁰. Evidence of monocytic potential of MLPs prompted us to test whether these cells can give rise to macrophages and DCs via a common intermediate. We seeded single CB MLPs on OP9 stroma, which supports myeloid, but not B or T cell differentiation at a clonal level. Single cells were first expanded into colonies with ‘primitive-acting’ cytokines and then matured into macrophages with M-CSF and IL-6 or DCs with GM-CSF and IL-4. As expected, M-CSF cultures were largely composed of CD14⁺CD11c⁺CD1a⁻ macrophages, whereas GM-CSF cultures contained CD14⁻CD11c⁺CD1a⁺ immature DCs (FIG. 12C, left panel). To investigate their combined macrophage and DC (MDC) potential, MLPs were cultured with both sets of cytokines (M-CSF, GM-CSF, IL-6 and IL-4). Over 45% of single CD7⁻ MLPs gave rise to colonies under these conditions, consistent with the cloning efficiency of myeloid progenitors (FIG. 12C, right panel). Of these, 78% contained both macrophages and DC progeny (FIG. 12C). suggesting that MLPs have a combined macropahge and DC potential.

### MLPs are the Primary Source of DCs

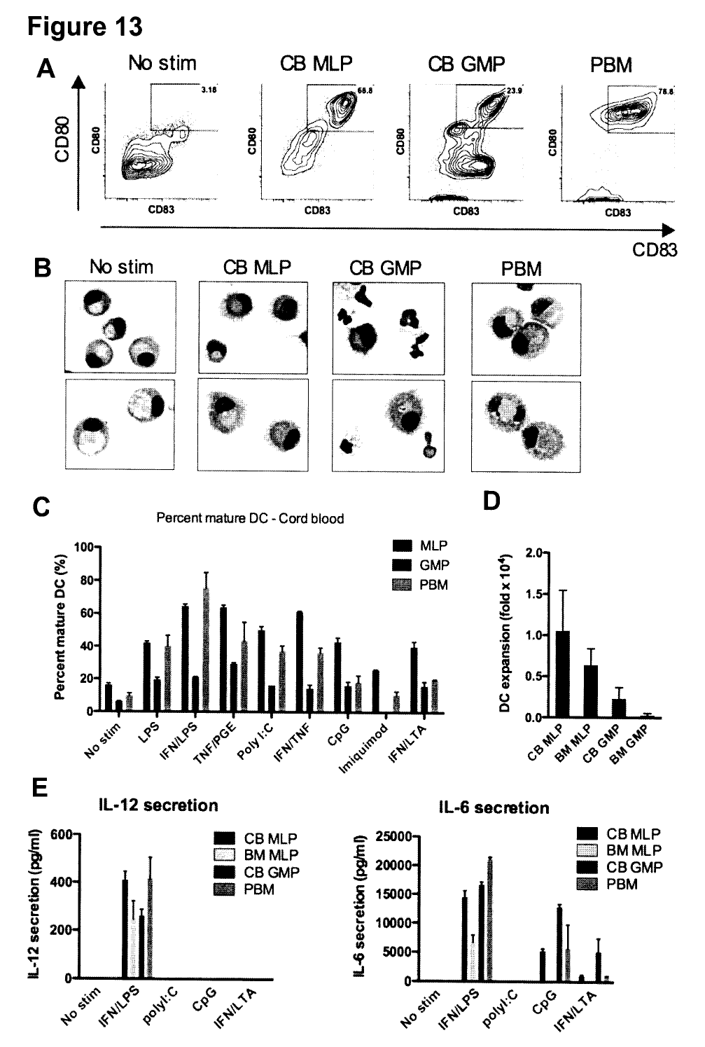

Previous studies suggested that while DCs could arise from both human lymphoid and myeloid progenitors, the myeloid pathway represented the primary source of DCs³¹. To investigate the potential of MLPs and myeloid progenitors to give rise to mature DCs, sorted MLPs or GMPs were expanded on OP9 stroma, differentiated into immature DCs with GM-CSF and IL-4, and matured by exposure to Toll-like receptor (TLR) ligands²⁹. These cells were compared to ‘standard’ DCs derived from CD14⁺ peripheral blood monocytes (PBMs). Mature DCs that upregulated HLA-DR, CD40, maturation marker CD83 and co-stimulatory molecules CD80 and CD86, were readily generated in a TLR-dependent manner (FIG. 13A,B). Various TLR stimulations differentiated MLPs into mature DCs more efficiently (up to 65% DC) than GMPs (up to 30% DC) or unfractionated CD34⁺ cells³², whose output consisted mostly of other myeloid cell types (FIG. 13C and data not shown). Using this protocol, a CB MLP yielded >10⁴ DCs compared with ˜10³ for GMP (FIG. 13D). DCs derived from all fractions secreted IL-12 involved in activation of cytotoxic T cells³³, IL-6, TNFa, and low levels of IL-10 (FIG. 13E13D). Thus, at least in vitro, MLPs represent a more potent source of DCs as compared to myeloid progenitors, and are thus suitable as a source for large-scale immune therapy applications.

### In Vivo MLPs Potential

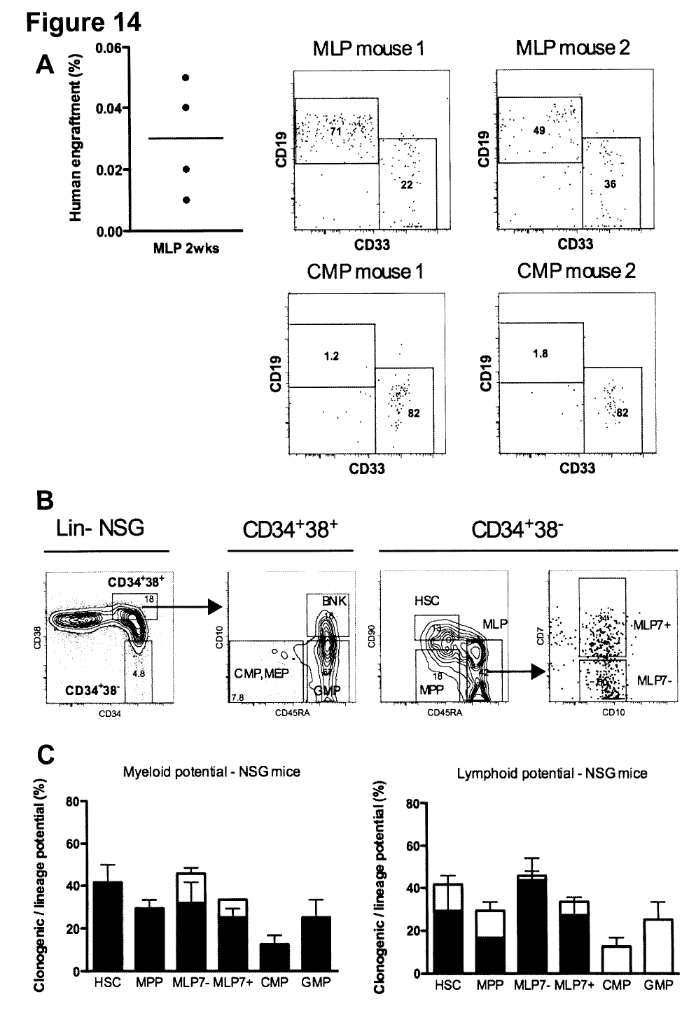

To determine the lineage potential of MLPs in vivo, we injected a near-limiting dose of 1,000 CB MLPs or CMPs directly into the femur of NOD-SCID-γ_{c} Null (NSG) mice and analyzed the composition of the graft after 2 and 4 weeks. CMPs gave rise to CD33⁺CD19⁻ myeloid grafts at 2 weeks in all recipients tested (FIG. 14A). However, by 4 weeks the remaining myeloid cells were at or below the limit of detection (0.01%; data not shown). These data indicated that the myeloid output of progenitors in NSG mice peaks at 2 weeks and declines thereafter. Transplanted CB MLPs (n=4) gave rise to grafts containing both CD19⁺ B cells and CD33⁺ myeloid cells at 2 weeks (FIG. 14A). The myeloid graft was substantially reduced at 4 weeks, consistent with the kinetics of myeloid output (data not shown). No T cells were detected, since MLPs only generated a transient graft in the injected femur, and T cell development requires long-term engraftment (Notta et al. manuscript in preparation). Notably, of the MLP-derived myeloid cells, we detected CD14⁺ monocytes, but not CD15⁺ granulocytes (data not shown). These data indicate that MLPs possess a bi-potent lympho-monocytic potential in vivo.

### Human HSCs Regenerate Progenitor Hierarchy

To determine if the progenitor classes we identified were generated de novo from HSCs, we analyzed the composition of the progenitor compartment in NSG mice stably repopulated by CB HSCs. Each of the 7 progenitor fractions identified in CB and BM including CD34⁺CD38⁻ Thy1⁴¹° CD45RA⁺ MLPs were faithfully reconstituted by transplanted HSCs (FIG. 14B). Moreover, the developmental potential of each fraction isolated from NSG mice was identical to those in CB or BM, as determined by clonal analysis on MS-5 stroma supplemented with SCF, TPO, IL-7 and IL-2. In particular, as for CB MLPs2B, every colony generated by CD7⁺ and CD7⁻ MLPs contained B or NK lymphoid progeny, and 70% of colonies also contained myeloid cells (FIG. 14C; cloning efficiency=45% and 34%, respectively). These results indicate that MLPs and other progenitors isolated from steady-state CB and BM are intrinsic components of the human hematopoietic tree derived from HSCs.

### Transcriptional Program of Human Progenitors

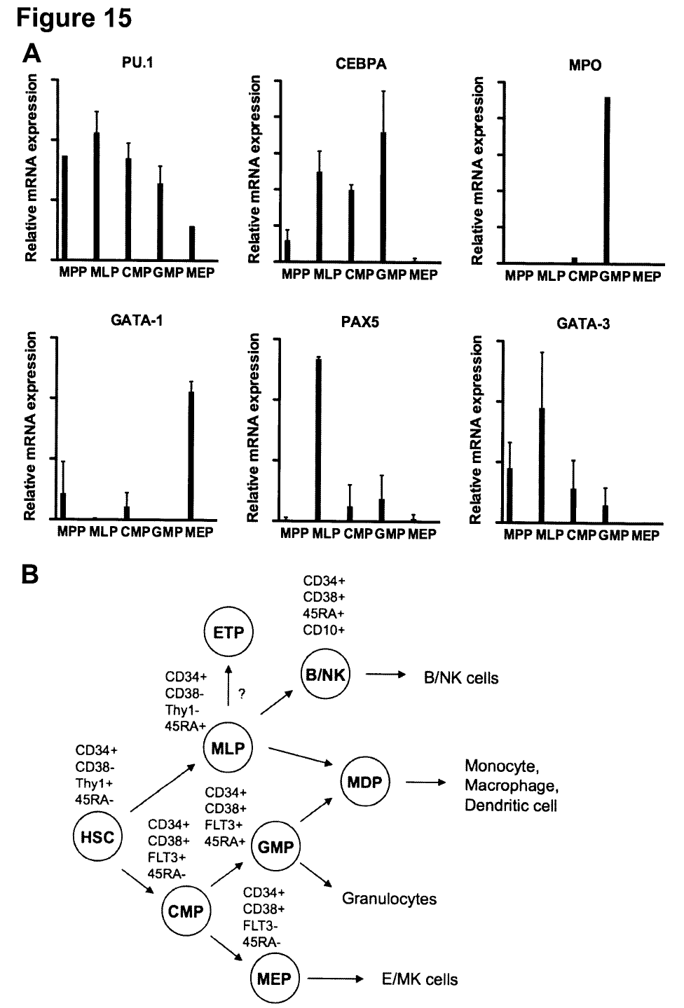

To investigate the transcriptional program that underlies human progenitor development, we performed quantitative PCR (qPCR) for lineage-specific markers (FIG. 15A), as their detection in uncommitted progenitors would be indicative of lineage potential ³⁴. SPI1 and CEBPA, which encode early myeloid transcription factors PU.1 and C/EBPα, were expressed in myeloid progenitors and also in MLPs. By contrast, the enzyme myeloperoxidase (MPO) produced by mature myeloid cells was only detected in GMPs. GATA-1, an erythroid master regulator, was selectively expressed in MEPs. Lastly, the key lymphoid transcription factors, PAXS and GATA-3, were selectively expressed in MLPs (FIG. 15A). Thus, the expression of lineage markers in progenitors correlated with their functional potential providing an independent line of evidence to support the proposed hierarchical organization.

This conclusion was further supported by global gene expression profiling. MLPs differentially expressed a set of annotated lymphoid genes as compared to multi-potent (HSC-MPP, p=3.2×10⁻⁵), myeloid (CMP; p=3.9×10⁻⁷), and erythroid (MEP; p=5.8×10⁻¹¹) progenitors. This gene signature included LY96, SYK, LTB, MIST, MHC class I and II and Ig loci. To obtain a signature of lineage-specific gene expression in MLPs, we used MEPs as a reference population for the MLP-enriched gene set, excluding stem cell-specific transcripts. The resulting set of 392 genes displayed two distinct expression patterns. A set of MLP-specific genes included LY96, SYK, LTB, MIST, LST1, MHC loci, and lymphoid transcription factors BCL6, BCL11A, NOTCH3 (FIG. 12D). A distinct cluster expressed by MLPs, GMPs, and CMPs, but not MPPs or MEPs, indicated a shared expression pattern between myeloid progenitors and MLPs (FIG. 12D). This set included myeloid transcription factors CEBPA and SPI1 (FIG. 15A), genes associated with innate immunity: IFITM1, LILRA2, INFGR1, CLEC4A, ITGB2, CCL3, and transcription factors IRF7 and IRF8 critical for development of Mφ and DCs³⁵. These results suggest that MLPs initiate expression of lymphoid transcripts, but maintain a shared gene expression signature with myeloid progenitors.

### Discussion

The present findings reveal the first comprehensive picture of early fate determination in human hematopoiesis (FIG. 15B). Applicants found that myeloid commitment followed the classical model, with the loss of lymphoid potential at the CMP stage, and the segregation of myeloid and erythroid potentials in GMPs and MEPs, respectively. Myeloid and E-MK potentials in the mouse were recently found to segregate to distinct cells within the CMP fraction³⁶, and this remains a possibility in human hematopoiesis. By contrast, human multi-lymphoid progenitors are not lymphoid-restricted, but give rise to dendritic cells and macrophages, in sharp opposition to the classical model. MLPs can be uniquely identified as Thy1^{-/lo}CD45RA⁺ cells within the immature CD34⁺CD38⁻ compartment in both CB and BM that also harbors Thy1⁺CD45RA⁻ HSCs and Thy1⁻CD45RA⁻ candidate MPPs²². In Applicants' assays, a high proportion of single cells within the MLP population gave rise to all the lymphoid and myelo-monocytic, but not erythroid or granulocytic lineages. Thus, human early lymphoid development involves a previously unknown lineage choice between the canonical lymphoid B, T, and NK cell fates, and MDC lineages traditionally viewed as myeloid-restricted. Applicants propose that the products of the MLP lineage choice in the bone marrow are the restricted B-NK precursors described here and MDC precursors, such as the MDP³⁰ .

The identification of MLPs extends the findings of two previous reports of human early lymphoid progenitors. The CD34⁺CD10⁺CD24⁻ phenotype¹⁸ is shared by MLPs and more mature progenitors, such as the B-NK precursors. The CD34⁺CD38⁻ CD7⁺ phenotype^{19, °}is more restrictive, because only half of CB MLPs are CD7⁺, and these cells are not found in adult BM. The precise phenotypic identification of human MLPs, combined with improved clonal assays, allowed us to interrogate their lineage potential at a single cell level. While previous reports detected only a residual myeloid potential, consistent with the classical model, we show that under improved conditions 57% of MLPs produced colonies on MS-5 stroma, and 85% of these contained B-NK and MDC lineages. Moreover, the ratio of myeloid, B cell, and NK outputs was nearly equal, indicating that these lineages are derived from the same cell. At least 45% of MLPs also generated T cells on OP9-DL1 stroma. Thus, it is most likely that this fraction contains a progenitor with combined B, T, NK, and MDC potential. These data and Applicants' survey of other progenitor populations provide no evidence for a lymphoid-restricted state (i.e. a CLP) in human hematopoiesis. It is currently believed that a CLP represents an obligate lymphoid intermediate in mouse, despite reports that myeloid potential is retained even after B-T-lineage restrictionl^{10, 12, 13}. Human MLPs do not give rise to granulocytes in vitro or in vivo and have a low repopulating capacity suggesting that they are also distinct from murine MLPPs. Reports of macrophage potential in murine and human ETP^{13, 37}, CLP³⁸ and the B-macrophage progenitors³⁹ support the notion that in mouse, as in human, macrophages may also arise in early lymphoid development.

Applicants' results also establish that the CD34⁺CD38⁻ Thy1^{-/lo}CD45RA⁺ phenotype identifies MLPs in both CB and BM. Known differences between neonatal and adult cells, such as the requirement for IL-7 in lymphopoiesis⁴⁰ gave rise to speculations that early lymphoid progenitors in CB and BM might be phenotypically and functionally distinct. However, the frequency and the B lymphoid, NK, and MDC lineage potentials of neonatal and adult human MLPs were comparable. Thus, the data strongly support the applicability of the proposed human hierarchy model to both neonatal and adult hematopoiesis. There are differences between adult and neonatal MLP in terms of the decreased capacity to generate T lymphocytes and their capacity to be instructed to myeloid fate by cytokines. Concordant with these data, the output of murine CLPs, ETPs and pro-B cells decreases with age²⁷ suggesting that age-related defects in immunity in mouse and human are in part attributed to the function of lymphoid progenitors.

MLPs give rise to B cells and monocytes upon transplantation into NSG mice, however it remains to be determined if MLPs contribute to the steady-state monocyte pool in humans. Primary monocytopenia is a rare disorder which is accompanied in some cases by B-NK cytopenias, with a severe depletion of circulating B, NK, and MDC cells, but normal hematocrit, neutrophil, and platelet counts⁴¹. Analysis of the CD34⁺ compartment in the bone marrow of one such patient revealed that CD34⁺CD38⁻Thy1⁺ HSCs and all progenitor populations were present, except the MLPs and the more committed B-NK precursors (Bigley et al. manuscript under submission). These observations suggest that MLP may be an obligate intermediate in human steady-state B-NK and MDC development. Notably, T cell development was affected to a lesser extent, suggesting that in humans, as in mice, many different progenitor populations can contribute to thymopoiesis⁴².

Monocytes, macrophages, and DCs belong to a network of immune cells termed the mononuclear phagocyte system, and share a common progenitor, the MDP^{30, 43}. Macrophages specialize in phagocytosis and innate immunity, while DCs specialize in antigen presentation to shape adaptive immune responses⁴⁴. DCs arise from both myeloid and lymphoid progenitors, while monocytes and macrophages were thought to arise uniquely from myeloid progenitors, such as GMPs⁴⁵. Our findings place the origin of MDC lineages in early human lymphopoiesis, revealing an intriguing redundancy in hematopoietic development that supports a version of the ‘myeloid-based’ model of hematopoiesis^{46, 47}.

DCs have a potent capacity to present antigens and stimulate T cells making them useful tools for immune therapy applications^{48, 49}. Since MLPs can be readily isolated from patient CB, mPB, or BM biopsies, expanded and differentiated to obtain large quantities of autologous T cells and DCs, they provide an attractive platform for tailoring immunotherapies for research purposes and for ongoing immune therapy trials.

Although preferred embodiments of the invention have been described herein, it will be understood by those skilled in the art that variations may be made thereto without departing from the spirit of the invention or the scope of the appended claims. All references disclosed herein, including those in the following reference list, are incorporated in their entirety by reference.

TABLE 1

| # | Phenotype | Name | Freq (% MNC) | Lineage output |
|---|---|---|---|---|
| — | CD34⁺CD38⁻Thy1⁺CD45RA⁻Flt3⁺CD7⁻CD10⁻ | HSC | 0.04 | All* |
| A | CD34⁺CD38⁻Thy1⁻CD45RA⁻Flt3⁺CD7⁻CD10⁻ | MPP | 0.04 | All* |
| B | CD34⁺CD38⁻Thy1 [symbol] CD45RA⁺Flt3⁺CD7⁻CD10⁺ | MLP7− | 0.01 | B, T, NK, MDC |
| C | CD34⁺CD38⁻Thy1⁻CD45RA⁺Flt3⁺CD7⁺CD10⁺ | MLP7+ | 0.01 | B, T, NK, MDC |
| D | CD34⁺CD38⁺Thy1⁻CD45RA⁻Flt3⁺CD7⁻CD10⁻ | CMP | 0.15 | EMK, G, MDC |
| E | CD34⁺CD38⁺Thy1⁻CD45RA⁺Flt3⁺CD7⁻CD10⁻ | GMP | 0.05 | G, MDC |
| F | CD34⁺CD38⁺Thy1⁻CD45RA⁻Flt3⁻CD7⁻CD10⁻ | MEP | 0.30 | EMK |
| G | CD34⁺CD38⁺Thy1⁻CD45RA⁺Flt3⁺CD7⁻CD10⁺ | B/NK | 0.05 | B or NK |

[symbol]

indicates data missing or illegible when filed

The list of candidate progenitor fractions sorted from CB and BM based on the 7-color flow cytometric analysis using the indicated combinations of cell surface markers. The flow cytometric representation of these populations is shown in FIG. 10. For each fraction, the fraction # (A-G), its full phenotype, functional designation, frequency (as % of CB mononuclear cells), and lineage output are indicated. Legend: B, B-cell; T, T-cell; NK, natural killer cell; MDC, macrophage and dendritic cell; G, granulocyte; EMK, erythroid and megakaryocyte; nd, not detected. *Multipotency of HSC and MPP fractions was demonstrated in vivo (Notta et al. manuscript in preparation).

TABLE 2

| | | | Phenotype of cells in wells | | |
|---|---|---|---|---|---|
| Cells per | # | positive | | Lymphoid | Myelo-lymphoid |
| well | wells | wells | Myeloid | (B, N, BN) | (MB, MN, MBN) |

Fraction B (CD34⁺CD38⁻Thy1⁻CD45RA⁺CD10⁺CD7⁻) Cord Blood

| | | | | | |
|---|---|---|---|---|---|
| 4 | 12 | 9 (75%) | 1 | 4 (0, 1, 3) | 4 (0, 1, 3) |
| 2 | 36 | 14 (39%) | 0 | 7 (1, 4, 2) | 7 (4, 1, 2) |
| 1 | 96 | 18 (19%) | 0 | 6 (2, 2, 2) | 12 (2, 3, 7) |
| Total | 144 | 41 | 1 | 17 (43%) | 23 (57%) |

1.0 myeloid:1.1 B-cell:1.3 NK cell

Fraction C (CD34⁺CD38⁻Thy1⁻CD45RA⁺CD10⁺CD7⁺) Cord Blood

| | | | | | |
|---|---|---|---|---|---|
| 5 | 24 | 10 (42%) | 0 | 4 (0, 1, 3) | 6 (1, 0, 5) |
| 2 | 24 | 4 (17%) | 0 | 4 (2, 1, 1) | 0 (0, 0, 0) |
| 1 | 36 | 4 (11%) | 0 | 1 (0, 0, 1) | 3 (0, 1, 2) |
| Total | 84 | 18 | 0 | 9 (50%) | 9 (50%) |

1.0 myeloid:1.7 B-cell:1.7 NK-cell

Fraction B (CD34⁺CD38⁻Thy1⁻CD45RA⁺CD10⁺CD7⁻) Bone Marrow

| | | | | | |
|---|---|---|---|---|---|
| 4 | 24 | 15 (58%) | 1 | 8 (2, 3, 3) | 6 (3, 0, 3) |
| 1 | 48 | 13 (27%) | 1 | 6 (2, 4, 0) | 6 (0, 4, 2) |
| Total | 72 | 28 | 2 | 14 (50%) | 12 (43%) |

1.0 myeloid:1.1 B-cell:1.4 NK cell

Limiting dilution analysis of candidate human MLP fractions on MS-5 stroma. The indicated number of cells from fractions B and C isolated from CB and BM (fraction C is not found in BM) were deposited by flow sorting into individual wells with MS-5 stroma and cultured for 4 wks with SCF, TPO, IL-7, and IL-2. Myeloid, lymphoid, or myelo-lymphoid colonies of 7 different subtypes (FIG. 2A11A), were identified using a panel of lineage markers, as described in the text and Methods. Colony counts were pooled from 2 or more independent experiments, with 12 or more wells per fraction each. Colony types representing >90% of total output for each fraction are shaded to indicate the likely lineage output. Legend: cell per well, number of cells deposited into each well; # wells, total number of wells seeded; positive wells, number of wells containing human cells; phenotype of cells in wells, number of wells containing cells of indicated lineage. Colony types are listed in parenthesis: B cell (B), NK cell (N), B and NK (BN), myeloid and B cell (MB); myeloid and NK cell (MN); myeloid, B, and NK (MBN). The ratios of lineage output (bottom row for each fraction) were calculated as: myeloid=number of M+MB+MN+MBN colonies; B lymphoid=number of B+BN+MB+MBN colonies; NK lymphoid=number of N+BN+MN+MBN colonies.

### References:

1. Iwasaki, H. & Akashi, K. Hematopoietic developmental pathways: on cellular basis. Oncogene 26, 6687-6696 (2007).

2. Dick, J. E. Stem cell concepts renew cancer research. Blood 112, 4793-4807 (2008).

3. Kondo, M., Weissman, I. L. & Akashi, K. Identification of clonogenic common lymphoid progenitors in mouse bone marrow. Cell 91, 661-672 (1997).

4. Akashi, K., Traver, D., Miyamoto, T. & Weissman, I. L. A clonogenic common myeloid progenitor that gives rise to all myeloid lineages. Nature 404, 193-197 (2000).

5. Adolfsson, J. et al. Identification of Flt3+lympho-myeloid stem cells lacking erythro-megakaryocytic potential a revised road map for adult blood lineage commitment. Cell 121, 295-306 (2005).

6. Mansson, R. et al. Molecular evidence for hierarchical transcriptional lineage priming in fetal and adult stem cells and multipotent progenitors. Immunity 26, 407-419 (2007).

7. Lai, A. Y. & Kondo, M. Asymmetrical lymphoid and myeloid lineage commitment in multipotent hematopoietic progenitors. J Exp Med 203, 1867-1873 (2006).

8. Igarashi, H., Gregory, S. C., Yokota, T., Sakaguchi, N. & Kincade, P. W. Transcription from the RAG1 locus marks the earliest lymphocyte progenitors in bone marrow. Immunity 17, 117-130 (2002).

9. Martin, C. H. et al. Efficient thymic immigration of B220+ lymphoid-restricted bone marrow cells with T precursor potential. Nat Immunol 4, 866-873 (2003).

10. Lu, M., Kawamoto, H., Katsube, Y., Ikawa, T. & Katsura, Y. The common myelolymphoid progenitor: a key intermediate stage in hemopoiesis generating T and B cells. J Immunol 169, 3519-3525 (2002).

11. Katsura, Y. Redefinition of lymphoid progenitors. Nat Rev Immunol 2, 127-132 (2002).

12. Bell, J. J. & Bhandoola, A. The earliest thymic progenitors for T cells possess myeloid lineage potential. Nature 452, 764-767 (2008).

13. Wada, H. et al. Adult T-cell progenitors retain myeloid potential. Nature 452, 768-772 (2008).

14. Bhandoola, A., von Boehmer, H., Petrie, H. T. & Zuniga-Pflucker, J. C. Commitment and developmental potential of extrathymic and intrathymic T cell precursors: plenty to choose from. Immunity 26, 678-689 (2007).

15. Weiner, R. S., Pelayo, R. & Kincade, P. W. Evolving views on the genealogy of B cells. Nat Rev Immunol 8, 95-106 (2008).

16. Manz, M. G., Miyamoto, T., Akashi, K. & Weissman, I. L. Prospective isolation of human clonogenic common myeloid progenitors. Proc Natl Acad Sci U S A 99, 11872-11877 (2002).

17. Galy, A., Travis, M., Cen, D. & Chen, B. Human T, B, natural killer, and dendritic cells arise from a common bone marrow progenitor cell subset. Immunity 3, 459-473 (1995).

18. Six, E. M. et al. A human postnatal lymphoid progenitor capable of circulating and seeding the thymus. J Exp Med 204, 3085-3093 (2007).

19. Hao, Q. L. et al. Identification of a novel, human multilymphoid progenitor in cord blood. Blood 97, 3683-3690 (2001).

20. Hoebeke, I. et al. T-, B- and NK-lymphoid, but not myeloid cells arise from human CD34(+)CD38(−)CD7(+) common lymphoid progenitors expressing lymphoid-specific genes. Leukemia 21, 311-319 (2007).

21. Yoshikawa, Y. et al. A clonal culture assay for human cord blood lymphohematopoietic progenitors. Hum Immunol 60, 75-82 (1999).

22. Majeti, R., Park, C. Y. & Weissman, I. L. Identification of a hierarchy of multipotent hematopoietic progenitors in human cord blood. Cell Stem Cell 1, 635-645 (2007).

23. La Motte-Mohs, R. N., Herer, E. & Zuniga-Pflucker, J. C. Induction of T cell development from human cord blood hematopoietic stem cells by Delta-like 1 in vitro. Blood (2004).

24. Reya, T. et al. A role for Wnt signalling in self-renewal of haematopoietic stem cells. Nature 423, 409-414 (2003).

25. Delaney, C. et al. Notch-mediated expansion of human cord blood progenitor cells capable of rapid myeloid reconstitution. Nat Med 16, 232-236.

26. Ng, S. Y., Yoshida, T., Zhang, J. & Georgopoulos, K. Genome-wide lineage-specific transcriptional networks underscore Ikaros-dependent lymphoid priming in hematopoietic stem cells. Immunity 30, 493-507 (2009).

27. Linton, P. J. & Dorshkind, K. Age-related changes in lymphocyte development and function. Nat Immunol 5, 133-139 (2004).

28. Leon, B., Lopez-Bravo, M. & Ardavin, C. Monocyte-derived dendritic cells formed at the infection site control the induction of protective T helper 1 responses against Leishmania. Immunity 26, 519-531 (2007).

29. Krutzik, S. R. et al. TLR activation triggers the rapid differentiation of monocytes into macrophages and dendritic cells. Nat Med 11, 653-660 (2005).

30. Fogg, D. K. et al. A clonogenic bone marrow progenitor specific for macrophages and dendritic cells. Science 311, 83-87 (2006).

31. Chicha, L., Jarrossay, D. & Manz, M. G. Clonal type I interferon-producing and dendritic cell precursors are contained in both human lymphoid and myeloid progenitor populations. J Exp Med 200, 1519-1524 (2004).

32. Arrighi, J. F., Hauser, C., Chapuis, B., Zubler, R. H. & Kindler, V. Long-term culture of human CD34(+) progenitors with FLT3-ligand, thrombopoietin, and stem cell factor induces extensive amplification of a CD34(−)CD 14(−) and a CD34(−)CD14(+) dendritic cell precursor. Blood 93, 2244-2252 (1999).

33. Trinchieri, G. Interleukin-12 and the regulation of innate resistance and adaptive immunity. Nat Rev Immunol 3, 133-146 (2003).

34. Miyamoto, T. et al. Myeloid or lymphoid promiscuity as a critical step in hematopoietic lineage commitment. Dev Cell 3, 137-147 (2002).

35. Wang, H. & Morse, H. C., 3rd IRF8 regulates myeloid and B lymphoid lineage diversification. Immunol Res 43, 109-117 (2009).

36. Pronk, C. J. et al. Elucidation of the phenotypic, functional, and molecular topography of a myeloerythroid progenitor cell hierarchy. Cell Stem Cell 1, 428-442 (2007).

37. Hao, Q. L. et al. Human intrathymic lineage commitment is marked by differential CD7 expression: identification of CD7− lympho-myeloid thymic progenitors. Blood 111, 1318-1326 (2008).

38. Balciunaite, G., Ceredig, R., Massa, S. & Rolink, A. G. A B220+ CD117+ CD19− hematopoietic progenitor with potent lymphoid and myeloid developmental potential. Eur J Immunol 35, 2019-2030 (2005).

39. Montecino-Rodriguez, E., Leathers, H. & Dorshkind, K. Bipotential B-macrophage progenitors are present in adult bone marrow. Nat Immunol 2, 83-88 (2001).

40. Payne, K. J. & Crooks, G. M. Immune-cell lineage commitment: translation from mice to humans. Immunity 26, 674-677 (2007).

41. Vinh, D. C. et al. Autosomal dominant and sporadic monocytopenia with susceptibility to mycobacteria, fungi, papillomaviruses, and myelodysplasia. Blood 115, 1519-1529.

42. Saran, N. et al. Multiple extrathymic precursors contribute to T-cell development with different kinetics. Blood 115, 1137-1144.

43. van Furth, R. & Cohn, Z. A. The origin and kinetics of mononuclear phagocytes. J Exp Med 128, 415-435 (1968).

44. Auffray, C., Sieweke, M. H. & Geissmann, F. Blood monocytes: development, heterogeneity, and relationship with dendritic cells. Annu Rev Immunol 27, 669-692 (2009).

45. Geissmann, F. et al. Development of monocytes, macrophages, and dendritic cells. Science 327, 656-661.

46. Kawamoto, H. & Katsura, Y. A new paradigm for hematopoietic cell lineages: revision of the classical concept of the myeloid-lymphoid dichotomy. Trends Immunol 30, 193-200 (2009).

47. Kawamoto, H. A close developmental relationship between the lymphoid and myeloid lineages. Trends Immunol 27, 169-175 (2006).

48. Melief, C. J. Cancer immunotherapy by dendritic cells Immunity 29, 372-383 (2008).

49. Tacken, P. J., de Vries, I. J., Torensma, R. & Figdor, C. G. Dendritic-cell immunotherapy: from ex vivo loading to in vivo targeting. Nat Rev Immunol 7, 790-802 (2007).

50. S. Doulatov et al., Nat Immunol 11, 585 (Jul).

51. R. Majeti, C. Y. Park, I. L. Weissman, Cell Stem Cell 1, 635 (Dec 13, 2007).

52. J. L. McKenzie, K. Takenaka, O. I. Gan, M. Doedens, J. E. Dick, Blood 109, 543 (Jan. 15, 2007).

53. Morrison, S. J., Wandycz, A. M., Hemmati, H. D., Wright, D. E. & Weissman, I. L. Identification of a lineage of multipotent hematopoietic progenitors. Development 124, 1929-1939 (1997).

54. Bertoncello, I., Hodgson, G. S. & Bradley, T. R. Multiparameter analysis of transplantable hemopoietic stem cells: I. The separation and enrichment of stem cells homing to marrow and spleen on the basis of rhodamine-123 fluorescence. Exp Hematol 13, 999-1006 (1985).

55. Spangrude, G. J. & Johnson, G. R. Resting and activated subsets of mouse multipotent hematopoietic stem cells. Proc Natl Acad Sci U S A 87, 7433-7437 (1990).

56. McKenzie, J. L., Takenaka, K., Gan, O. I., Doedens, M. & Dick, J. E. Low rhodamine 123 retention identifies long-term human hematopoietic stem cells within the Lin-CD34+CD38− population. Blood 109, 543-545 (2007).

1. A method for enriching a population of cells for human hematopoietic stem cells (HSCs) comprising:
identifying and providing the population of cells that is a source of HSCs and is to be enriched for HSCs; and
sorting cells in the population by a level of CD49f expression.

2. The method of claim 1, further comprising dividing the cells into high, intermediate and low CD49f expression groups.

3. The method of claim 2, further comprising selecting for a sub-population of cells comprising at least one of the intermediate and high level CD49f expression groups.

4. The method of claim 2, further comprising selecting for a sub-population of cells comprising the high CD49f expression group.

5. The method of claim 1, further comprising sorting the cells by the level of Rhodamine-123 staining

6. The method of claim 5, further comprising dividing the cells into high and low Rhodamine-123 staining groups.

7. The method of claim 6, further comprising selecting cells comprising the low Rhodamine-123 staining group.

8. A method for enriching a population of cells for human hematopoietic stem cells (HSCs) comprising:
identifying and providing the population of cells that is a source of HSCs and is to be enriched for HSCs; and
sorting cells in the population by a level of Rhodamine-123 staining.

9. The method of claim 8, further comprising dividing the cells into high and low Rhodamine-123 staining groups.

10. The method of claim 9, further comprising selecting for a sub-population of cells comprising the low Rhodamine-123 staining group.

11. The method of claim 1, further comprising sorting the cells by the level of CD49f expression.

12. The method of claim 11, further comprising dividing the cells into high, intermediate and low CD49f expression groups.

13. The method of claim 12, further comprising selecting for cells comprising at least one of the intermediate and high level CD49f expression groups.

14. The method of claim 13, further comprising selecting for cells comprising the high CD49f expression group.

15. The method of claim 1; further comprising sorting cells using at least one marker selected from the group consisting of Lin, CD34, CD38, CD90, Thy1 and CD45RA.

16. The method of claim 15, further comprising selecting at least one fraction selected from the group consisting of Lin⁻, CD34⁺, CD38⁻, CD90⁺, Thy1⁺ and CD45RA⁻.

17. The method of claim 1, wherein the source of the population of cells is at least one of bone marrow, umbilical cord blood, mobilized peripheral blood, spleen or fetal liver.

18. A population of cells enriched for HSCs obtained by the method of claim 1.

19.-42. (canceled)
